(19) United States
(12) Patent Application Publication
(10) Pub. No.: US 2026/0327215 A1
(43) Pub. Date: Oct. 22, 2026

(54) ISOXAZOLIDINES AS RIPK1 INHIBITORS AND USE THEREOF

(71) Applicant: GENZYME CORPORATION, Cambridge, MA (US)

(72) Inventors: Elisabeth DEFOSSA, Frankfurt am Main (DE); Uwe HEINELT, Frankfurt am Main (DE); Nils RACKELMANN, Frankfurt am Main (DE); Maria MENDEZ PEREZ, Frankfurt am Main (DE); Hans MATTER, Frankfurt am Main (DE); Niels GRIESANG, Frankfurt am Main (DE); Matthias SCHIELL, Frankfurt am Main (DE); Jozsef PANCZEL, Frankfurt am Main (DE)

(21) Appl. No.: 19/104,262

(22) PCT Filed: Aug. 17, 2023
(86) PCT No.: PCT/US2023/072366

§ 371 Date: Feb. 17, 2025

(30) Foreign Application Priority Data
Aug. 19, 2022 (EP) 22315187.9

(51) Int. Cl. C07D 413/14; A61K 31/506; A61K 45/06

(52) U.S. Cl. CPC C07D 413/14; A61K 31/506; A61K 45/06

(57) ABSTRACT

Disclosed herein are isoxazolidine compounds and their use as receptor-interacting protein kinase 1 inhibitor, for example in the treatment of diseases and disorders mediated by RIP kinase 1.

### RELATED APPLICATIONS

The present invention claims the benefit of European Patent Application Serial No. 22315187.9, filed Aug. 19, 2022, the content of which is incorporated herein by reference in its entirety for all purposes.

### BACKGROUND

Although inflammation can be a protective mechanism in response to harmful stimuli such as invasion of pathogens and tissue damages, chronic inflammation is an important underlying factor in many human diseases such as neurodegeneration, rheumatoid arthritis, autoimmune and inflammatory diseases, and cancer. Similarly, the activation of cell death pathways, such as necrosis and apoptosis which are useful in eliminating infected or damaged cells, is also an important underlying mechanism for human diseases, including acute and chronic neurodegenerative diseases. Receptor-interacting protein kinase 1 (UniProtKB Q13546) is a key regulator of inflammation, apoptosis and necroptosis. Receptor-interacting protein kinase 1 has an important role in modulating inflammatory responses mediated by nuclear-factor kappa-light chain enhancer of activated B cells (NF-κB). More recent research has shown that its kinase activity controls necroptosis, a form of necrotic cell death, which was traditionally thought to be passive and unregulated, and is characterized by a unique morphology. Further, receptor-interacting protein kinase 1 is part of a pro-apoptotic complex indicating its activity in regulating apoptosis.

The receptor-interacting protein kinase 1 is subject to complex and intricate regulatory mechanisms, including ubiquitylation, deubiquitylation, and phosphorylation. These regulatory events collectively determine whether a cell will survive and activate an inflammatory response or die through apoptosis or necroptosis. Dysregulation of receptor-interacting protein kinase 1 signalling can lead to excessive inflammation or cell death, and conversely, research has shown that inhibition of receptor-interacting protein kinase 1 can be effective therapies for diseases involving inflammation or cell death.

RIPK1 inhibition has been identified as a promising principle to address different diseases like rheumatoid arthritis (RA), psoriasis, multiple sclerosis, Alzheimer's disease, inflammatory bowel disease such as Crohn's disease, amyotrophic lateral sclerosis (ALS) or ulcerative colitis (UC). To treat some of these diseases like multiple sclerosis (MS) and Alzheimer's disease, access to the central nervous system (CNS) is required, while for other diseases like rheumatoid arthritis, psoriasis, inflammatory bowel disease (IBD) such as Crohn's disease or UC access to the CNS is not essentially required.

Different RIPK1 inhibitors were already described (e.g. WO2014125444, WO2016185423), WO2016027253 (GSK).

The most advanced RIPK1 inhibitor, GSK2982772 (oxazepinone derivative disclosed in WO2014/125444), was evaluated for RA, psoriasis and UC in phase II clinical trials.

Dihydropyrazole compounds with phenyl substituent on dihydropyrazole and a pyrimidine-piperidine element are disclosed as RIPK1 inhibitors by GSK in WO2018092089.

Isoxazolidine compounds with phenyl substituent on isoxazolidine and a pyrimidine-piperidine element are disclosed as RIPK1 inhibitors by GSK in WO2019130230. Similar isoxazolidine compounds are disclosed in KR2020087922 (Voronoi) and in WO2020043173.

An isoxazolidine compound with phenyl substituent on isoxazolidine and methoxy substituted pyrimidine as RIPK1 inhibitor is disclosed in WO2021245070.

### SUMMARY

Disclosed herein are compounds of formula I:

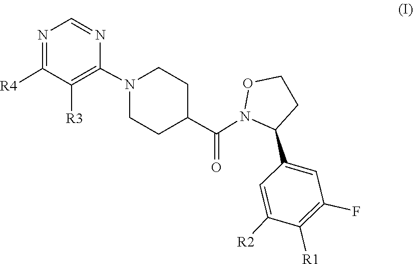

wherein
R1 is H or C₁-C₄ allyl,
R2 is CN or halogen,
R3 is H or halogen,
R4 is NH₂ or OCH₃,
   or a pharmaceutically acceptable salt, solvate, or stereoisomer thereof,
   wherein the compound of formula I with R1═H, R2=F, R3=H and R4=OCH₃ is excluded.

In another aspect provided are methods for making the compound and intermediates thereof.

In a related aspect, provided herein pharmaceutical composition comprising a compound as described herein and a pharmaceutically acceptable excipient.

In another aspect, provided herein is a method of inhibiting receptor-interacting protein kinase 1. Further provided are methods for treating receptor-interacting protein kinase 1-mediated disease or disorder comprising administering a therapeutically effective amount of a compound or a pharmaceutical composition as described herein to a subject in need thereof. The disclosure also provides uses of the compound or compositions thereof in the manufacture of a medicament for the treatment of a disease, disorder or condition that is mediated by (or mediated, at least in part, by) receptor-interacting protein kinase 1.

### DETAILED DESCRIPTION

### Definitions

As used herein chemical nomenclature as not defined otherwise have the meanings as being used in the technical field.

As used herein, the term “C₁-C₄-allyl” refers to an allyl group selected from methyl, ethyl, n-propyl, i-propyl, n-butyl, i-butyl and t-butyl.

As used herein, the term “halogen” refers to F, Cl, Br or I.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response or other problem or complication, commensurate with a reasonable benefit/risk ratio.

The term “pharmaceutically-acceptable salts” in this respect refers to the relatively non-toxic, inorganic and organic acid addition salts of compounds of the present disclosure. These salts can be prepared in situ in the administration vehicle or the dosage form manufacturing process or by separately reacting a purified compound of the disclosure in its free base form with a suitable organic or inorganic acid and isolating the salt thus formed during subsequent purification.

As used herein, the term “pharmaceutically acceptable excipient” refers to a substance that aids the administration of an active agent to a subject. By “pharmaceutically acceptable”, it is meant that the excipient is compatible with the other ingredients of the formulation and is not deleterious to the recipient thereof. Pharmaceutical excipients useful in the present disclosure include, but are not limited to, binders, fillers, disintegrants, lubricants, glidants, coatings, sweeteners, flavors and colors.

The phrase “pharmaceutically-acceptable carrier” as used herein means a pharmaceutically acceptable material, composition or vehicle, such as a liquid or solid filler, diluent, excipient or solvent encapsulating material, involved in carrying or transporting the subject compound from one organ or portion of the body, to another organ or portion of the body.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation, or experiment. The methods described herein may be useful in human therapy and/or veterinary applications. In some embodiments, the subject is a mammal. In one embodiment, the subject is a human.

The term “therapeutically effective amount” or “effective amount” of a compound described herein or a pharmaceutically acceptable salt, tautomer, stereoisomer, mixture of stereoisomers, prodrug, or deuterated analog thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition of as described herein. The therapeutically effective amount may vary depending on the subject, and disease or condition being treated, the weight and age of the subject, the severity of the disease or condition, and the manner of administering, which can readily be determined by one of ordinary skill in the art.

As used herein chemical nomenclature as not defined otherwise have the meanings as being used in the technical field.

Disclosed herein is a compound of formula I:

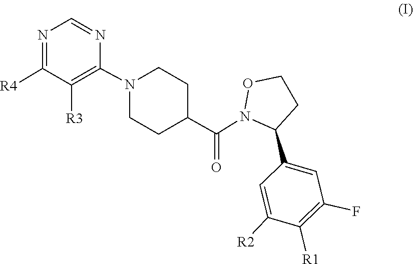

wherein
R1 is H or C₁-C₄ allyl,
R2 is CN or halogen,
R3 is H or halogen,
R4 is NH₂ or OCH₃,
   or a pharmaceutically acceptable salt, solvate, or stereoisomer thereof,
   wherein the compound of formula I with R1═H, R2=F, R3=H and R4=OCH₃ is excluded.

One embodiment are compounds of formula I,
wherein
R1 represents C₁-C₄ alkyl,
R2 represents CN;
or a pharmaceutically acceptable salt, solvate, or stereoisomer thereof.

One embodiment are compounds of formula I,
wherein
R1 represents H,
R2 represents halogen;
or a pharmaceutically acceptable salt, solvate, or stereoisomer thereof.

One embodiment are compounds of formula I,
wherein
R1 represents H,
R2 represents Cl;
or a pharmaceutically acceptable salt, solvate, or stereoisomer thereof.

One embodiment are compounds of formula I,
wherein
R1 represents CH₃,
R2 represents CN;
or a pharmaceutically acceptable salt, solvate, or stereoisomer thereof.

One embodiment are compounds of formula I,
wherein
R1 represents H,
R2 represents CN;
or a pharmaceutically acceptable salt, solvate, or stereoisomer thereof.

One embodiment are compounds of formula I,
wherein
R1 represents H,
R2 represents F;
or a pharmaceutically acceptable salt, solvate, or stereoisomer thereof.

One embodiment are compounds of formula I,
R3 represents H,
or a pharmaceutically acceptable salt, solvate, or stereoisomer thereof.

One embodiment are compounds of formula I,
R3 represents halogen,
or a pharmaceutically acceptable salt, solvate, or stereoisomer thereof.

One embodiment are compounds of formula I,
R3 represents F,
or a pharmaceutically acceptable salt, solvate, or stereoisomer thereof.

One embodiment are compounds of formula I,
R4 represents NH₂,
or a pharmaceutically acceptable salt, solvate, or stereoisomer thereof.

One embodiment are compounds of formula I,
R4 represents OCH₃,
or a pharmaceutically acceptable salt, solvate, or stereoisomer thereof.

One embodiment are compounds of formula I,
wherein
R1 represents H,
R2 represents CN,
R3 represents H or F;
R4 represents NH₂ or OCH₃,
or a pharmaceutically acceptable salt, solvate, or stereoisomer thereof.

One embodiment are compounds of formula I,
wherein
R1 represents H,
R2 represents CN,
R3 represents H;
R4 represents NH₂ or OCH₃,
or a pharmaceutically acceptable salt, solvate, or stereoisomer thereof.

One embodiment are compounds of formula I,
wherein
R1 represents H,
R2 represents CN,
R3 represents F;
R4 represents NH₂ or OCH₃,
or a pharmaceutically acceptable salt, solvate, or stereoisomer thereof.

One embodiment are compounds of formula I,
wherein
R1 represents H,
R2 represents F,
R3 represents F;
R4 represents NH₂ or OCH₃,
or a pharmaceutically acceptable salt, solvate, or stereoisomer thereof.

One embodiment are compounds of formula I,
wherein
when R2 is halogen, then R4 is NH₂,
or a pharmaceutically acceptable salt, solvate, or stereoisomer thereof.

One embodiment are compounds of formula I,
wherein
R2 is CN,
or a pharmaceutically acceptable salt, solvate, or stereoisomer thereof.

Another embodiment are compounds of formula I, selected from
3-[(3S)-2-[1-(6-Aminopyrimidin-4-yl)piperidine-4-carbonyl]isoxazolidin-3-yl]-5-fluoro-benzonitrile,
[1-(6-aminopyrimidin-4-yl)-4-piperidyl]-[(3S)-3-(3,5-difluorophenyl)isoxazolidin-2-yl]methanone,
5-[(3S)-2-[1-(6-aminopyrimidin-4-yl)piperidine-4-carbonyl]isoxazolidin-3-yl]-3-fluoro-2-methyl-benzonitrile,
(S)-3-fluoro-5-(2-(1-(6-methoxypyrimidin-4-yl)piperidine-4-carbonyl)isoxazolidin-3-yl)benzonitrile,
3-fluoro-5-[(3S)-2-[1-(5-fluoro-6-methoxy-pyrimidin-4-yl)piperidine-4-carbonyl]isoxazolidin-3-yl]benzonitrile,
3-fluoro-5-[(3S)-2-[1-(6-methoxypyrimidin-4-yl)piperidine-4-carbonyl]isoxazolidin-3-yl]-2-methyl-benzonitrile and [(3S)-3-(3-chloro-5-fluoro-phenyl)isoxazolidin-2-yl]-[1-(6-methoxypyrimidin-4-yl)-4-piperidyl]methanone,
   or a pharmaceutically acceptable salt, solvate, or stereoisomer thereof.

Another embodiment are compounds of formula I, selected from
3-[(3S)-2-[1-(6-Aminopyrimidin-4-yl)piperidine-4-carbonyl]isoxazolidin-3-yl]-5-fluoro-benzonitrile,
[1-(6-aminopyrimidin-4-yl)-4-piperidyl]-[(3S)-3-(3,5-difluorophenyl)isoxazolidin-2-yl]methanone and
5-[(3S)-2-[1-(6-aminopyrimidin-4-yl)piperidine-4-carbonyl]isoxazolidin-3-yl]-3-fluoro-2-methyl-benzonitrile,
   or a pharmaceutically acceptable salt, solvate, or stereoisomer thereof.

Another embodiment are compounds of formula I, selected from
(S)-3-fluoro-5-(2-(1-(6-methoxypyrimidin-4-yl)piperidine-4-carbonyl)isoxazolidin-3-yl)benzonitrile,
3-fluoro-5-[(3S)-2-[1-(5-fluoro-6-methoxy-pyrimidin-4-yl)piperidine-4-carbonyl]isoxazolidin-3-yl]benzonitrile,
3-fluoro-5-[(3S)-2-[1-(6-methoxypyrimidin-4-yl)piperidine-4-carbonyl]isoxazolidin-3-yl]-2-methyl-benzonitrile and
[(3S)-3-(3-chloro-5-fluoro-phenyl)isoxazolidin-2-yl]-[1-(6-methoxypyrimidin-4-yl)-4-piperidyl]methanone,
   or a pharmaceutically acceptable salt, solvate, or stereoisomer thereof.

### Pharmaceutical Compositions

The compounds provided herein is usually administered in the form of pharmaceutical compositions. Thus, provided herein are also pharmaceutical compositions that contain one or more of the compounds described herein or a pharmaceutically acceptable salt, tautomer, stereoisomer, mixture of stereoisomers, prodrug, or deuterated analog thereof and one or more pharmaceutically acceptable vehicles selected from carriers, adjuvants and excipients.

Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical arts.

The pharmaceutical compositions of the present disclosure may be specially formulated for administration in solid or liquid form, including those adapted for the following: oral administration, for example, drenches (aqueous or non-aqueous solutions or suspensions), tablets, e.g., those targeted for buccal, sublingual and systemic absorption, boluses, powders, granules, pastes for application to the tongue; parenteral administration, for example, by subcutaneous, intramuscular, intravenous, or epidural injection as, for example, a sterile solution or suspension or sustained-release formulation; topical application, for example, as a cream, ointment or a controlled-release patch or spray applied to the skin; intravaginally or intrarectally, for example, as a pessary, cream or foam; sublingually; ocularly; transdermally; or nasally, pulmonary and to other mucosal surfaces.

Some examples of materials which can serve as pharmaceutically-acceptable carriers include: sugars, such as lactose, glucose and sucrose; starches, such as corn starch and potato starch; cellulose and its derivatives, such as sodium carboxymethyl cellulose, ethyl cellulose and cellulose acetate; surfactants, such as polysorbate 80 (i.e., Tween 80); powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations. Examples of such formulations include, but are not limited to DMSO, 10 mM DMSO, 8% hydroxypropyl-beta-cyclodextrin in PBS, propylene glycol, etc. For example, in a certain embodiment the compounds of the disclosure can be used as 4 mM solution in 8% hydroxypropyl-beta-cyclodextrin in PBS for parenteral administration. In another certain embodiments, the compounds of the disclosure can be used as a suspension in 0.5% aqueous CMC containing 0.1% TWEEN 80.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions. Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol and the like; and metal chelating agents, such as citric acid, ethylenediamine tetraacetic acid (EDTA), sorbitol, tartaric acid, phosphoric acid and the like.

Formulations of the present disclosure include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, this amount will range from about 1% to about 99% of active ingredient, preferably from about 5% to about 70%, most preferably from about 10% to about 30%. In certain embodiments, a formulation of the present disclosure comprises one or more of cyclodextrins, liposomes, micelle forming agents, e.g., bile acids and polymeric carriers, e.g., polyesters and polyanhydrides; and a compound of the present disclosure. In certain embodiments, an aforementioned formulation renders orally bioavailable a compound of the present disclosure.

Methods of preparing these formulations or compositions include the step of bringing into association a compound of the present disclosure with the carrier and, optionally, one or more accessory ingredients. In general, the formulations are prepared by uniformly and intimately bringing into association a compound of the present disclosure with liquid carriers or finely divided solid carriers or both and then, if necessary, shaping the product. Formulations of the disclosure suitable for oral administration may be in the form of capsules, cachets, pills, tablets, lozenges (using a flavored basis, usually sucrose and acacia or tragacanth), powders, granules or as a solution or a suspension in an aqueous or non-aqueous liquid or as an oil-in-water or water-in-oil liquid emulsion or as an elixir or syrup or as pastilles (using an inert base, such as gelatin and glycerin or sucrose and acacia) and/or as mouth washes and the like, each containing a predetermined amount of a compound of the present disclosure as an active ingredient. A compound of the present disclosure may also be administered as a bolus, electuary or paste.

In solid dosage forms of the disclosure for oral administration (capsules, tablets, pills, dragees, powders, granules and the like), the active ingredient is mixed with one or more pharmaceutically-acceptable carriers, such as sodium citrate or dicalcium phosphate and/or any of the following: fillers or extenders, such as starches, lactose, sucrose, glucose, mannitol and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates and sodium carbonate; solution retarding agents, such as paraffin; absorption accelerators, such as quaternary ammonium compounds; wetting agents, such as, for example, cetyl alcohol, glycerol monostearate and non-ionic surfactants; absorbents, such as kaolin and bentonite clay; lubricants, such a talc, calcium stearate, magnesium stearate, solid polyethylene glycols, sodium lauryl sulfate and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent.

The tablets and other solid dosage forms of the pharmaceutical compositions of the present disclosure, such as dragees, capsules, pills and granules, may optionally be scored or prepared with coatings and shells, such as enteric coatings and other coatings well known in the pharmaceutical formulating arts. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter or by incorporating sterilizing agents in the form of sterile solid compositions that can be dissolved in sterile water or some other sterile injectable medium immediately before use. These compositions may also optionally contain opacifying agents and may be of a composition that they release the active ingredient(s) only or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth and mixtures thereof.

Formulations of the pharmaceutical compositions of the disclosure for rectal or vaginal administration may be presented as a suppository, which may be prepared by mixing one or more compounds of the disclosure with one or more suitable nonirritating excipients or carriers comprising, for example, cocoa butter, polyethylene glycol, a suppository wax or a salicylate and which is solid at room temperature, but liquid at body temperature and, therefore, will melt in the rectum or vaginal cavity and release the active compound.

Formulations of the present disclosure which are suitable for vaginal administration also include pessaries, tampons, creams, gels, pastes, foams or spray formulations containing such carriers as are known in the art to be appropriate.

Dosage forms for the topical or transdermal administration of a compound of this disclosure include powders, sprays, ointments, pastes, creams, lotions, gels, solutions, patches and inhalants. The active compound may be mixed under sterile conditions with a pharmaceutically-acceptable carrier and with any preservatives, buffers or propellants which may be required.

The ointments, pastes, creams and gels may contain, in addition to an active compound of this disclosure, excipients, such as animal and vegetable fats, oils, waxes, paraffins, starch, tragacanth, cellulose derivatives, polyethylene glycols, silicones, bentonites, silicic acid, talc and zinc oxide or mixtures thereof.

Powders and sprays can contain, in addition to a compound of this disclosure, excipients such as lactose, talc, silicic acid, aluminum hydroxide, calcium silicates and polyamide powder or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present disclosure to the body. Dissolving or dispersing the compound in the proper medium can make such dosage forms. Absorption enhancers can also be used to increase the flux of the compound across the skin. Either providing a rate controlling membrane or dispersing the compound in a polymer matrix or gel can control the rate of such flux.

Ophthalmic formulations, eye ointments, powders, solutions and the like, are also contemplated as being within the scope of this disclosure. Pharmaceutical compositions of this disclosure suitable for parenteral administration comprise one or more compounds of the disclosure in combination with one or more pharmaceutically-acceptable sterile isotonic aqueous or nonaqueous solutions, dispersions, suspensions or emulsions or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers, which may be employed in the pharmaceutical compositions of the disclosure include water, ethanol, polyols (such as glycerol, propylene glycol, polyethylene glycol and the like) and suitable mixtures thereof, vegetable oils, such as olive oil and injectable organic esters, such as ethyl oleate. Proper fluidity can be maintained, for example, by the use of coating materials, such as lecithin, by the maintenance of the required particle size in the case of dispersions and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenyl sorbic acid and the like. It may also be desirable to include isotonic agents, such as sugars, sodium chloride and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form. Alternatively, delayed absorption of a parenterally-administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

### Methods of Treatment

In other embodiments, provided herein is a method of treating a receptor-interacting protein kinase 1-mediated disease or disorder. The method includes administering a therapeutically effective amount of a compound or pharmaceutical composition as described herein to a subject in need thereof. In some embodiments, the receptor-interacting protein kinase 1-mediated disease or disorder is trauma, ischemia, stroke, cardiac infarction, infection, Gaucher's disease, Krabbe disease, sepsis, systemic inflammatory response syndrome (SIRS), Parkinson's disease, Alzheimer's disease, amyotrophic lateral sclerosis (ALS), multiple sclerosis (MS), Huntington's disease, HIV-associated dementia, retinal degenerative disease, glaucoma, age-related macular degeneration, rheumatoid arthritis, non-communicable inflammatory skin diseases (ncISD) such as psoriasis or atopic dermatitis, psoriatic arthritis, or inflammatory bowel disease.

The term “trauma” as used herein refers to any physical damage to the body caused by violence, accident, fracture etc. The term “ischemia” refers to a cardiovascular disorder characterized by a low oxygen state usually due to the obstruction of the arterial blood supply or inadequate blood flow leading to hypoxia in the tissue. The term “stroke” refers to cardiovascular disorders caused by a blood clot or bleeding in the brain, most commonly caused by an interruption in the flow of blood in the brain as from clot blocking a blood vessel and in certain embodiments of the disclosure the term stroke refers to ischemic stroke or hemorrhagic stroke. The term “myocardial infarction” refers to a cardiovascular disorder characterized by localized necrosis resulting from obstruction of the blood supply.

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals.

Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art. The selected compounds may be further characterized to examine the safety or tolerance dosage in human or non-human subjects. Such properties may be examined using commonly known methods to those skilled in the art.

Experiments with knockout animal models and Necrostatin 1, a receptor-interacting protein kinase 1 inhibitor, have demonstrated the effectiveness of receptor-interacting protein kinase 1 inhibition in protecting tissues from inflammatory bowel diseases (e.g., ulcerative colitis and Crohn's disease), non-communicable inflammatory skin diseases (ncISD) such as psoriasis or atopic dermatitis, retinal-detachment-induced photoreceptor necrosis, retinitis pigmentosa, cerulein-induced acute pancreatitis, and sepsis/systemic inflammatory response syndrome (SIRS), and alleviating ischemic brain injury, retinal ischemia/reperfusion injury, Huntington's disease, renal ischemia reperfusion injury, cisplatin induced kidney injury, traumatic brain injury, hematological and solid organ malignancies, bacterial infections and viral infections (e.g., tuberculosis and influenza or SARS-Coronavirus) and lysosomal storage diseases. The receptor-interacting protein kinase 1 inhibitors of the present disclosure are therefore suggesting that both RIPK1 kinase-driven inflammation and cell death are key contributing factors to systemic inflammatory response syndrome (SIRS). There is also rationale that vascular permeability and endothelial dysfunction contribute to SIRS/shock and lethality. The receptor-interacting protein kinase 1 inhibitors of the present disclosure are therefore useful for treating diseases and conditions mediated by receptor-interacting protein kinase 1, including but not limited to inflammatory diseases or disorders, necrotic cell diseases, neurodegenerative diseases, central nervous system (CNS) diseases, ocular diseases, infections, and malignancies. In certain embodiments, the receptor-interacting protein kinase 1 inhibitors described herein can inhibit inflammation, protect tissue or cell from damage or undesired cell death (e.g., necrosis or apoptosis), ameliorate symptoms, and improve immune response or neuronal function in a patient suffering from any of the prescribed diseases or conditions. Moreover, the compounds may be suitable for treatment of immune-mediated disease, such as but not limited to, allergic diseases, autoimmune diseases, and prevention of transplant rejection.

Provided herein are compounds and compositions for use in medicine. In certain embodiments, the compounds and compositions are for use in the treatment of a receptor-interacting protein kinase 1-mediated disease or disorder. Also provided is a method of treating a receptor-interacting protein kinase 1-mediated disease or disorder comprising administering a therapeutically effective amount of a compound or pharmaceutical composition disclosed herein to a subject in need thereof. In certain embodiments, the disease or disorder is an inflammatory disease associated with A20 SNPs.

In some embodiments, the compounds and compositions are for use in the treatment of Alzheimer disease, multiple sclerosis, amyotrophic lateral sclerosis (ALS) and incontinentia pigmenti.

Various specific diseases and disorders are described below. In certain embodiments, the disease or disorder is necrotizing enterocolitis, tuberous sclerosis, Tangier's Disease, Wohlman's Syndrome, inflammatory bowel disease, Crohn's disease, ulcerative colitis, non-communicable inflammatory skin diseases (ncISD) such as psoriasis or atopic dermatitis, retinal detachment, retinitis pigmentosa, macular degeneration, pancreatitis (e.g., acute pancreatitis), interface dermatitis (e.g. cutaneous lupus erythematosus, lichen planus, lichen planopillaris, toxic epidermal necrolysis (TEN), Stevens-Johnson-Syndrome, Graft versus Host Disease (GvHD), alopecia arreata, vitiligo), atopic dermatitis, rheumatoid arthritis, spondyloarthritis, gout, SoJIA, systemic lupus erythematosus, Sjogren's syndrome, systemic scleroderma, anti-phospholipid syndrome, vasculitis, osteoarthritis, non-alcohol steatohepatitis, alcohol steatohepatitis, autoimmune hepatitis autoimmune hepatobiliary diseases, primary sclerosing cholangitis, nephritis, Celiac disease, autoimmune ITP, transplant rejection, ischemia reperfusion injury of solid organs, sepsis, systemic inflammatory response syndrome (SIRS), cerebrovascular accident, myocardial infarction, Huntington's disease, Alzheimer's disease, Parkinson's disease, allergic diseases, asthma, atopic dermatitis, multiple sclerosis, type I diabetes, Wegener's granulomatosis, pulmonary sarcoidosis, Behçet's disease, interleukin-1 converting enzyme associated fever syndrome, chronic obstructive pulmonary disease, tumor necrosis factor receptor-associated periodic syndrome, periodontitis, bacterial infection, staphylococcus infection, mycobacterium infection, retinitis pigmentosa, influenza, severe acute respiratory syndrome (SARS), middle east respiratory syndrome (MERS), acute respiratory response syndrome (ARDS), transplant rejection, burns or hypoxia. In certain embodiments, the disease or disorder is trauma, ischemia, stroke, cardiac infarction, infection, lysosomal storage disease, Niemann-Pick disease, Gaucher's disease, Krabbe disease, sepsis, systemic inflammatory response syndrome (SIRS), Parkinson's disease, Alzheimer's disease, amyotrophic lateral sclerosis (ALS), Huntington's disease, HIV-associated dementia, encephalopathy, retinal degenerative disease, glaucoma, age-related macular degeneration, rheumatoid arthritis, non-communicable inflammatory skin diseases (ncISD) such as psoriasis or atopic dermatitis, psoriatic arthritis or inflammatory bowel disease. In certain embodiments, the disease or disorder is Alzheimer's disease, ALS, Friedreich's ataxia, Huntington's disease, Lewy body disease, Parkinson's disease, Huntington's disease, multiple sclerosis, diabetic neuropathy, polyglutamine (polyQ) diseases, stroke, Fahr disease, Menke's disease, Wilson's disease, cerebral ischemia, lysosomal storage disease or a prion disorder. In certain embodiments, the disease is ALS. In certain embodiments, the disease is Alzheimer's disease. In certain embodiments, the disease is lysosomal storage disease. In certain embodiments, the disease is Parkinson's disease. In certain embodiments the disorder is an ischemic disease of organs including but not limited to brain, heart, kidney and liver. In some different embodiments, the disorder is an ocular disorder such as retinal degenerative disease, glaucoma or age-related macular degeneration. In some different embodiments, the disorder is a central nervous system (CNS) disorder.

In certain embodiments, provided is a method of treating rheumatoid arthritis (see Lawlor KE, Nat Commun. 2015, 6282, Lee S H, Sci Rep. 2017, 10133), systemic onset juvenile idiopathic arthritis (SoJIA), spondyloarthritis, osteoarthritis, non-communicable inflammatory skin diseases (ncISD) such as psoriasis or atopic dermatitis, Crohn's disease, ulcerative colitis, or multiple sclerosis, comprising administering a therapeutically effective amount of a compound as provided herein to a subject in need thereof. In certain embodiments, provided is a method of treating autoimmune hepatitis, atherosclerosis, neutrophilic dermatoses, or a rare disease driven by A20, NEMO, and/or LUBAC mutations, comprising administering a therapeutically effective amount of a compound as provided herein to a subject in need thereof.

In certain embodiments, the compounds and compositions are useful for treating non-communicable inflammatory skin diseases (ncISD) such as psoriasis or atopic dermatitis.

In certain embodiments, the disorder is an inflammatory disease of the intestines such as Crohn's disease or ulcerative colitis (both generally known together as inflammatory bowel disease, IBD). In certain embodiments, the mammal is a primate, canine or feline subject. In certain embodiments, the mammal is a human subject. While not wishing to be bound by theory, it is believed that inhibition of receptor-interacting protein kinase 1 by the presently disclosed compounds is responsible, at least in part, for their anti-inflammatory activity. Accordingly, embodiments of the disclosure also include methods for inhibiting receptor-interacting protein kinase 1, either in vitro or in a subject in need thereof, the method comprises contacting a receptor-interacting protein kinase 1 with a compound disclosed herein. In some of these embodiments, inhibiting receptor-interacting protein kinase 1 is effective to block (partially or fully) the release of inflammatory mediators such as TNF and/or IL6.

### Inflammatory Diseases or Disorders

The receptor-interacting protein kinase 1 inhibitors described herein may be used to treat inflammatory diseases and disorders. Inflammatory diseases and disorders typically exhibit high levels of inflammation in the connective tissues or degeneration of these tissues.

Non-limiting examples of inflammatory diseases and disorders include Alzheimer's disease, ankylosing spondylitis, arthritis including osteoarthritis, rheumatoid arthritis (RA), non-communicable inflammatory skin diseases (ncISD) such as psoriasis or atopic dermatitis, asthma, atherosclerosis, Crohn's disease, colitis, dermatitis, diverticulitis, fibromyalgia, hepatitis, irritable bowel syndrome (IBS), inflammatory bowel disease (IBD), systemic lupus erythematous (SLE), nephritis, Parkinson's disease and ulcerative colitis. In certain embodiments, the compounds and compositions of the present disclosure are useful for treating an autoimmune disorder, such as rheumatoid arthritis, psoriasis, psoriatic arthritis, encephalitis, allograft rejection, autoimmune thyroid diseases (such as Graves' disease and Hashimoto's thyroiditis), autoimmune uveoretinitis, giant cell arteritis, inflammatory bowel diseases (including Crohn's disease, ulcerative colitis, regional enteritis, granulomatous enteritis, distal ileitis, regional ileitis, and terminal ileitis), insulin-dependent diabetes mellitus, multiple sclerosis, pernicious anemia, sarcoidosis, scleroderma, and systemic lupus erythematosus. In an embodiment, the receptor-interacting protein kinase 1 inhibitors described herein are useful for treating autoimmune encephalitis. In certain embodiments, the compounds and compositions are useful for treating rheumatoid arthritis (RA). In certain embodiments, the compounds and compositions are useful for treating ulcerative colitis. In certain embodiments, the compounds and compositions are useful for treating non-communicable inflammatory skin diseases (ncISD) such as psoriasis or atopic dermatitis.

In certain embodiments, the disorder is an inflammatory disease of the intestines such as Crohn's disease or ulcerative colitis (both generally known together as inflammatory bowel disease). In certain embodiments, the mammal is a primate, canine or feline subject. In certain embodiments, the mammal is a human subject. While not wishing to be bound by theory, it is believed that inhibition of receptor-interacting protein kinase 1 by the presently disclosed compounds is responsible, at least in part, for their anti-inflammatory activity. Accordingly, embodiments of the disclosure also include methods for inhibiting receptor-interacting protein kinase 1, either in vitro or in a subject in need thereof, the method comprises contacting a receptor-interacting protein kinase 1 with a compound disclosed herein. In some of these embodiments, inhibiting receptor-interacting protein kinase 1 is effective to block (partially or fully) the release of inflammatory mediators such as TNF and/or IL6.

In another embodiment the receptor-interacting protein kinase 1 inhibitors described herein may be used to treat inflammatory diseases and disorders like rheumatoid arthritis (RA), psoriasis, inflammatory bowel disease (IBD) like Crohn's disease or ulcerative colitis.

In another embodiment the receptor-interacting protein kinase 1 inhibitors described herein may be used to treat Interface dermatitis like cutaneous lupus erythematosus (CLE), Lichen planus (LP), toxic epidermal necrolysis (TEN) or Stevens-Johnson syndrome (SJS).

In another embodiment the receptor-interacting protein kinase 1 inhibitors described herein may be used to treat hyperinflammation during viral infection like corona virus disease-19 (COVID-19), acute respiratory distress syndrome (ARDS), systemic inflammatory response syndrome (SIRS).

In another embodiment the receptor-interacting protein kinase 1 inhibitors described herein may be used to treat corona virus disease-19 (COVID-19).

In another embodiment the receptor-interacting protein kinase 1 inhibitors described herein may be used to treat Respiratory diseases like Influenza (e.g. swine flu, H7N9), severe acute respiratory syndrome (SARS), Middle East Respiratory Syndrome (MERS), or Respiratory-Syncytial-Virus (RSV) or bronchiolitis).

### Necrotic Cell Diseases

The compounds described herein may be used for the treatment of diseases/disorders caused or otherwise associated with cellular necrosis. In particular, the disclosure provides methods for preventing or treating a disorder associated with cellular necrosis in a mammal, comprising the step of administering to said mammal a therapeutically effective amount of a compound or composition described herein.

The term “necrotic cell disease” refers to diseases associated with or caused by cellular necrosis, for example trauma, ischemia, stroke, cardiac infarction, infection, Gaucher's disease, Krabbe disease, sepsis, systemic inflammatory response syndrome (SIRS), Parkinson's disease, Alzheimer's disease, amyotrophic lateral sclerosis, Huntington's disease, HIV-associated dementia, retinal degenerative disease, glaucoma, age-related macular degeneration, rheumatoid arthritis, non-communicable inflammatory skin diseases (ncISD) such as psoriasis or atopic dermatitis, psoriatic arthritis or inflammatory bowel disease.

The necrotic cell diseases can be acute diseases such as trauma, ischemia, stroke, cardiac infarction, anthrax lethal toxin induced septic shock, sepsis, systemic respiratory response syndrome (SIRS), cell death induced by LPS and HIV induced T-cell death leading to immunodeficiency. In certain embodiments the disorder is an ischemic disease of organs including but not limited to brain, heart, kidney and liver.

The necrotic cell diseases also include chronic neurodegenerative diseases, such as Parkinson's disease, Huntington's disease, amyotrophic lateral sclerosis, Alzheimer's disease, infectious encephalopathies, dementia such as HIV associated dementi. In some different embodiments, the disorder is an ocular disorder such as retinal degenerative disease, glaucoma or age-related macular degeneration. In some different embodiments, the disorder is a central nervous system (CNS) disorder.

### Neurodegenerative and CNS Diseases

The receptor-interacting protein kinase 1 inhibitors described herein may also be used to treat neurodegenerative diseases. Neurodegenerative diseases can affect many of the body's activities, such as balance, movement, talking, breathing, and heart function. Neurodegenerative diseases can be genetic or caused by medical conditions such as alcoholism, tumors, strokes, toxins, chemicals, and viruses. Non-limiting examples of neurodegenerative diseases include Alzheimer's disease, amyotrophic lateral sclerosis (ALS), Friedreich's ataxia, Huntington's disease, Lewy body disease, Parkinson's disease, and spinal muscular atrophy. In certain embodiments, neurodegenerative diseases and CNS diseases include Niemann-Pick disease, type C1 (NPC1), Alzheimer's disease, amyotrophic lateral sclerosis (ALS), Friedreich's ataxia, Huntington's disease, Lewy body disease, Parkinson's disease, and spinal muscular atrophy.

In certain embodiments, the receptor-interacting protein kinase 1 inhibitors described herein may be used to treat NPC1 via inhibiting necroptosis that causes neuronal loss. In certain embodiments, the compounds and compositions of the present disclosure are useful for treating Alzheimer's disease. In certain embodiments, the compounds and compositions of the present disclosure are useful for treating Parkinson's disease. In certain embodiments, the compounds and compositions of the present disclosure are useful for treating amyotrophic lateral sclerosis (ALS).

More generally, the receptor-interacting protein kinase 1 inhibitors described herein can be used to preserve neuron viability and promote axon growth and nerve functions within the central nervous system (CNS). Accordingly, the compounds may be used to reduce or even reverse the loss of cognitive, motor, and sensory functions associated with a CNS disease or disorder, by preserving neuron viability and/or promoting axon regeneration and/or nerve functions.

The receptor-interacting protein kinase 1 inhibitors described herein can be used in a method for promoting axon regeneration in a CNS neuron, such as a CNS sensory neuron, a motor neuron, a cortical neuron, a cerebellar neuron, a hippocampal neuron, and a midbrain neuron. The receptor-interacting protein kinase 1 inhibitors described herein can be used in a method for promoting nerve function or preserving the viability following injury to a CNS neuron. In another embodiments, these compounds can be used to promote regeneration of an axon in a CNS neuron that is degenerated in the CNS disease or disorder. The receptor-interacting protein kinase 1 inhibitors may be administered by any conventional means, such as locally to the neuron or applied ex vivo before re-implantation.

Accordingly, in one aspect, the disclosure provides a method of treating a CNS disorder in a subject in need thereof, wherein a symptom of the CNS disorder is axon degeneration or injury within a CNS neuron. The method comprises administering to the subject an effective amount of a compound or composition disclosed herein thereby to promote regeneration of an axon in a CNS neuron affected by the CNS disorder. Following administration, neural functions may be measured, for example, as an indication of axon regeneration. It is also contemplated that, following administration of a compound or composition, the neuron function of the CNS neuron is preserved or improved relative to the neuron function prior to administration.

Non-limiting examples of CNS diseases or disorders include brain injury, spinal cord injury, dementia, stroke, Alzheimer's disease, amyotrophic lateral sclerosis (ALS), Parkinson's disease, Huntington's disease, multiple sclerosis, diabetic neuropathy, polyglutamine (polyQ) diseases, stroke, Fahr disease, Menke's disease, Wilson's disease, cerebral ischemia, and a prion disorder.

In exemplary embodiments, the CNS disorder is brain injury or spinal cord injury.

Also provided herein are methods for promoting neuron survival and axon regeneration in the CNS. CNS disorders characterized by impaired or failing axon growth or axon degeneration may arise from CNS neuron injury (e.g., trauma, surgery, nerve compression, nerve contusion, nerve transection, neurotoxicity or other physical injury to the brain or spinal cord) or neurodegenerative CNS disease, wherein a symptom of the disorder is axon degeneration (e.g., Alzheimer's disease, amyotrophic lateral sclerosis (ALS), Parkinson's disease, multiple sclerosis, diabetic neuropathy, polyglutamine (polyQ) diseases, stroke, Fahr disease, Menke's disease, Wilson's disease, cerebral ischemia, prion disorder (e.g., Creutzfeldt-Jakob disease). In certain embodiments, the CNS disorder is brain injury (e.g., traumatic brain injury) or spinal cord injury (e.g., chronic, acute or traumatic spinal cord injury). In certain embodiments, the CNS disorder affects a subject's basic vital life functions such as breathing, heartbeat and blood pressure, e.g., an injury to or aneurysm in the brain stem. In certain embodiments, the CNS disease or disorder affects a subject's cognitive ability. In certain embodiments, the CNS disease or disorder affects a subject's movement and/or strength. In certain embodiments, the CNS disease or disorder affects a subject's coordination.

In certain embodiments, the CNS disorder affects a subject's cognitive ability, such as, brain injury to the cerebral cortex or a neurodegenerative CNS disorder, such as, Alzheimer's disease, frontotemporal dementia, dementia with Lewy bodies, corticobasal degeneration, progressive supranuclear palsy and prion disorders.

In certain embodiments, the CNS disorder affects a subject's movement and/or strength, such as injury to the brain or spinal cord or a neurodegenerative CNS disorder such as Parkinson's disease, frontotemporal dementia, dementia with Lewy bodies, corticobasal degeneration, progress supranuclear palsy, Huntington's disease, multiple system atrophy, amyotrophic lateral sclerosis and hereditary spastic paresis. In certain embodiments, the CNS disorder affects a subject's coordination, such as brain injury to the cerebellum or a neurodegenerative CNS disorder such as spinocerebellar atrophies, Friedreich's ataxia and prion disorders.

In each of the foregoing methods, the CNS disorder includes, but is not limited to, brain injury, spinal cord injury, Alzheimer's disease, amyotrophic lateral sclerosis (ALS), Parkinson's disease, multiple sclerosis, diabetic neuropathy, polyglutamine (polyQ) diseases, stroke, Fahr disease, Menke's disease, Wilson's disease, cerebral ischemia, a prion disorder (e.g., Creutzfeldt-Jakob disease), dementia (e.g., frontotemporal dementia, dementia with Lewy bodies), corticobasal degeneration, progressive supranuclear palsy, multiple system atrophy, hereditary spastic paraparesis and spinocerebellar atrophies.

Non-limiting examples of neurodegenerative diseases include Alzheimer's disease, lysosomal storage diseases, amyotrophic lateral sclerosis (ALS), Friedreich's ataxia, Huntington's disease, Lewy body disease, Parkinson's disease, and spinal muscular atrophy.

In certain embodiments, the compounds and compositions of the present disclosure are useful for treating Alzheimer's disease. In certain embodiments, the compounds and compositions of the present disclosure are useful for treating Parkinson's disease. In certain embodiments, the compounds and compositions of the present disclosure are useful for treating amyotrophic lateral sclerosis (ALS). In certain embodiments, the compounds and compositions of the present disclosure are useful for treating lysosomal storage diseases.

In certain embodiments, the disorder is a brain disorders, such as, but not limited to, Alzheimer's disease, ALS, frontotemporal dementias, vascular dementia, Huntington's disease, Parkinson's disease, Lewy Body dementia, Progressive Supranuclear Palsy, multiple sclerosis, neuromyelitis optica, ischemic brain damage (stroke), hypoxic brain damage, traumatic brain injury, spinal cord injury, sepsis-induced brain damage, CNS infections, CNS abscesses, glioblastoma multiforme, epilepsy, neuropathic pain, major depression, bipolar depression, schizophrenia, autism, Niemann-Pick disease, neuro-Behçet's disease.

In certain embodiments, provided is a method of treating a CNS disease or disorder, comprising administering a therapeutically effective amount of a compound as provided herein to a subject in need thereof. In certain embodiments, the disease or disorder is Alzheimer's disease or amyotrophic lateral sclerosis (ALS).

### Ocular Conditions

The receptor-interacting protein kinase 1 inhibitors described herein can also be used to treat ocular conditions, for example to reduce or prevent the loss of photoreceptor and/or retinal pigment epithelial cell viability.

In certain embodiments, the disclosure provides a method of preserving the visual function of an eye of a subject with an ocular condition, wherein a symptom of the ocular condition is the loss of photoreceptor cell viability in the retina of the eye with the condition. The method comprises administering to the eye of the subject an effective amount of a compound or composition described herein, thereby preserving the viability of the photoreceptor cells disposed within the retina of the eye. After administration, the visual function (e.g., visual acuity) of the eye may be preserved or improved relative to the visual function of the eye prior to administration.

The ocular condition may be age-related macular degeneration (AMD), retinosis pigmentosa (RP), macular edema, diabetic retinopathy, central areolar choroidal dystrophy, BEST disease, adult vitelliform disease, pattern dystrophy, myopic degeneration, central serous retinopathy, Stargardt's disease, Cone-Rod dystrophy, North Carolina dystrophy, infectious retinitis, inflammatory retinitis, uveitis, toxic retinitis, or light-induced toxicity. AMD may be the neovascular or the dry form of AMD. Retinal detachment may be arhegmatogenous, a serous, and a tractional retinal detachment. In certain embodiments, the ocular condition may be geographic atrophy, glaucoma, or another ischemic eye disease. In certain embodiments, the disclosure provides a method of preserving the viability of retinal pigment epithelial (RPE) cells within the retina of a subject with an ocular condition with administration of a compound of the present disclosure. The subject being treated may have a loss of retinal pigment epithelial cells in the retina of the eye with the condition and the ocular condition may be age-related macular degeneration (AMD), BEST disease, myopic degeneration, Stargardt's disease, uveitis, adult foveomacular dystrophy, fundus falvimaculatus, multiple evanescent white dot syndrome, serpiginous choroidopathy, acute multifocal posterior placoid epitheliopathy (AMPPE), or another uveitis disorder. In certain embodiments, the method comprises administering to the eye of the subject an effective amount of a compound or composition described herein, thereby preserving the viability of the retinal pigment epithelial cells. Provided in another embodiment is a method of preserving the viability of photoreceptor cells disposed within a retina of a subject with age-related macular degeneration (AMD), retinosis pigmentosa (RP), macular edema, diabetic retinopathy, central areolar choroidal dystrophy, BEST disease, adult vitelliform disease, pattern dystrophy, myopic degeneration, central serous retinopathy, Stargardt's disease, Cone-Rod dystrophy, North Carolina dystrophy, infectious retinitis, inflammatory retinitis, uveitis, toxic retinitis, or light-induced toxicity. Therefore, in certain embodiments, the method comprises administering to the eye an effective amount of a compound or composition described herein, thereby preserving the viability of the photoreceptor cells disposed within the retina of the subject with a condition. Provided in another embodiment is a method of preserving the viability of photoreceptor cells disposed within a retina of a mammalian eye following retinal detachment. The retinal detachment may be a rhegmatogenous retinal detachment, tractional retinal detachment, or serous retinal detachment. In other embodiments, the retinal detachment may occur as a result of a retinal tear, retinoblastoma, melanoma or other cancers, diabetic retinopathy, uveitis, choroidal neovascularization, retinal ischemia, pathologic myopia, or trauma. In certain embodiments, the method comprises administering a compound or composition described herein to the eye in which a region of the retina has been detached in amounts sufficient to preserve the viability of photoreceptor cells disposed within the region of the detached retina. Provided in another embodiment is a method of preserving visual function of an eye of a subject with age-related macular degeneration (AMD), retinosis pigmentosa (RP), macular edema, central areolar choroidal dystrophy, retinal detachment, diabetic retinopathy, BEST disease, adult vitelliform disease, pattern dystrophy, myopic degeneration, central serous retinopathy, Stargardt's disease, Cone-Rod dystrophy, North Carolina dystrophy, infectious retinitis, inflammatory retinitis, uveitis, toxic retinitis, or light-induced toxicity, wherein a symptom of the ocular condition is the loss of photoreceptor cells viability in the retina of the eye, wherein the method comprises treating the subject with a compound or composition described herein to the subject. In another aspect, the disclosure provides a method of preserving the visual function of an eye of a subject with an ocular condition, wherein a symptom of the ocular condition is the loss of photoreceptor cell viability and/or RPE viability in the retina of the eye wherein the method comprises treating the subject with a compound or composition described herein to the subject.

In certain embodiments, provided a method of preserving the visual function of an eye of a subject with ocular conditions, wherein a symptom of the ocular condition is the loss of retinal ganglion cell viability in the retina of the eye with the conditions. The method comprises administering to the eye of the subject an effective amount of a compound or composition, thereby preserving the viability of the retinal ganglion cells disposed within the retina of the eye. After administration of the compound or composition, the visual function of the eye may be preserved or improved relative to the visual function of the eye prior to administration. Further, after the administration, the preserved retinal ganglion cell is capable of supporting axonal regeneration.

Non-limiting examples of symptoms associated with the ocular conditions include the loss of retinal ganglion cell viability in the retina of the eye, glaucoma, optic nerve injury, optic neuritis, optic neuropathies, diabetic retinopathy, central retinal artery occlusion, and central retinal vein occlusion. The compounds described herein may also be used for the treatment of optic neuropathies such as ischemic optic neuropathy (e.g., arteritic or non-arteritic anterior ischemic neuropathy and posterior ischemic optic neuropathy), compressive optic neuropathy, infiltrative optic neuropathy, traumatic optic neuropathy, mitochondrial opticneuropathy (e.g., Leber's optic neuropathy), nutritional optic neuropathy, toxic optic neuropathy, and hereditary optic neuropathy (e.g., Leber's optic neuropathy, Dominant Optic Atrophy, Behr's syndrome).

Also disclosed is a method of preserving the visual function of an eye of a subject with glaucoma, optic nerve injury, optic neuropathies, diabetic retinopathy, central retinal artery occlusion, or central retinal vein occlusion. The method comprises administering to the eye of the subject an effective amount of a compound or composition described herein, thereby preserving the viability of the retinal ganglion cells disposed within the retina of the eye and the visual function of the eye.

In another aspect, disclosed herein is a method of preserving the viability of retinal ganglion cells disposed within a retina of a mammalian eye affected by, for example, glaucoma, optic nerve injury, optic neuritis, optic neuropathies, diabetic retinopathy, central retinal artery occlusion and central retinal vein occlusion. The method comprises administering a compound or composition described herein to the eye in which a region of the retina has been affected in amounts sufficient to preserve the viability of retinal ganglioncells disposed within the region of the affected retina. The preserved retinal ganglion cell is capable of supporting axonal regeneration, linear ubiquitin chain assembly complex (LUBAC) deficiency syndrome, hematological and solid organ malignancies, bacterial infections and viral infections (e.g., tuberculosis and influenza or SARS-Coronavirus) and lysosomal storage diseases.

Non-limiting examples of lysosomal storage diseases include Gaucher disease, GM2 Gangliosidosis, alpha-mannosidosis, aspartylglucosaminuria, cholesteryl ester storage disease, chronic hexosaminidase A deficiency, cystinosis, Danon disease, Fabry disease, Farber disease, fucosidosis, galactosialidosis, GM1 gangliosidosis, mucolipidosis, infantile free sialic acid storage disease, juvenile hexosaminidase A deficiency, Krabbe disease, lysosomal acid lipase deficiency, metachromatic leukodystrophy, mucopolysaccharidoses disorders, multiple sulfatase deficiency, Niemann-Pick disease, neuronal ceroid lipofuscinoses, Pompe disease, pycnodysostosis, Sandhoff disease, Schindler disease, sialic acid storage disease, Tay-Sachs and Wolman disease. In certain embodiments, provided are compounds and compositions for use in medicine. In certain embodiments, the compounds and compositions are for use in the treatment of a receptor-interacting protein kinase 1-mediated disease or disorder. Also provided is a method of treating a receptor-interacting protein kinase 1-mediated disease or disorder comprising administering a therapeutically effective amount of a compound or pharmaceutical composition disclosed herein to a subject in need thereof. In another embodiment, the present disclosure provides a method of inhibiting receptor-interacting protein kinase 1. The method includes contacting the receptor-interacting protein kinase 1 with an effective amount of a compound as described herein. Inhibiting the receptor-interacting protein kinase 1 generally include contacting the receptor-interacting protein kinase 1 with an amount of a compound sufficient to reduce the activity of the receptor-interacting protein kinase 1 as compared to the receptor-interacting protein kinase 1 activity in the absence of a compound. For example, contacting the receptor-interacting protein kinase 1 with a compound can result in from about 1% to about 99% receptor-interacting protein kinase 1 inhibition (i.e., the activity of the inhibited enzyme ranges from 99% to 1% of the enzyme activity in the absence of a compound). The level of receptor-interacting protein kinase 1 inhibition can range from about 1% to about 10%, or from about 10% to about 20%, or from about 20% to about 30%, or from about 30% to about 40%, or from about 40% to about 50%, or from about 50% to about 60%, or from about 60% to about 70%, or from about 70% to about 80%, or from about 80% to about 90%, or from about 90% to about 99%. The level of receptor-interacting protein kinase 1 inhibition can range from about 5% to about 95%, or from about 10% to about 90%, or from about 20% to about 80%, or from about 30% to about 70%, or from about 40% to about 60%. In some embodiments, contacting the receptor-interacting protein kinase 1 with a compound as described herein will result in complete (i.e., 100%) inhibition.

### Combination Therapy

In certain embodiments, the compounds described herein may be administered in combination with at least one other therapeutically active agent. The two or more agents can be coadministered, coformulated, or administered separately.

In certain embodiments, the other therapeutically active agent is selected from a thrombolytic agent, a tissue plasminogen activator, an anticoagulant, a platelet aggregation inhibitor, an antimicrobial agent (an antibiotic, a broad-spectrum antibiotic, a lactam, an antimycobacterial agent, a bactericidal antibiotic, anti-MRSA therapy), a long acting beta agonist, a combination of an inhaled corticosteroid and a long acting beta agonist, a short acting beta agonist, a leukotriene modifier, an anti-IgE, a methylxanthine bronchodilator, a mast cell inhibitor, a protein tyrosine kinase inhibitor, a CRTH2/D prostanoid receptor antagonist, an epinephrine inhalation aerosol, a phosphodiesterase inhibitor, a combination of a phosphodiesterase-3 inhibitor and a phosphodiesterase-4 inhibitor, a long-acting inhaled anticholinergic, a muscarinic antagonist, a long-acting muscarinic antagonist, a low dose steroid, an inhaled corticosteroid, an oral corticosteroid, a topical corticosteroid, anti-thymocyte globulin, thalidomide, chlorambucil, a calcium channel blocker, a topical emollient, an ACE inhibitor, a serotonin reuptake inhibitor, an endothelin-I receptor inhibitor, an anti-fibrotic agent, a proton-pump inhibitor, a cystic fibrosis transmembrane conductance regulator potentiator, a mucolytic agent, pancreatic enzymes, a bronchodilator, an ophthalmic intravitreal injection, an anti-vascular endothelial growth factor inhibitor, a ciliary neurotrophic growth factor agent, a trivalent (IIV3) inactivated influenza vaccine, a quadrivalent (IIV4) inactivated influenza vaccine, a trivalent recombinant influenza vaccine, a quadrivalent live attenuated influenza vaccine, an antiviral agent, inactivated influenza vaccine, a ciliary neurotrophic growth factor, a gene transfer agent, a topical immunomodulator, calcineurin inhibitor, an interferon gamma, an antihistamine, a monoclonal antibody, a polyclonal anti-Tcell antibody, an anti-thymocyte gamma globulin-equine antibody, an antithymocyte globulin-rabbit antibody, an anti-CD40 antagonist, a JAK inhibitor, and an anti-TCR murine mAb.

A compound described herein may be administered in combination with other anti-inflammatory agents for any of the indications above, including oral or topical corticosteroids, anti-TNF agents, 5-aminosalicyclic acid and mesalamine preparations, hydroxycloroquine, thiopurines, methotrexate, cyclophosphamide, cyclosporine, calcineurin inhibitors, mycophenolic acid, mTOR inhibitors, JAK inhibitors, Syk inhibitors, anti-inflammatory biologic agents, including anti-IL6 biologics, anti-IL1 agents, anti-IL1 7 biologics, anti-CD22, anti-integrin agents, anti-IFNa, anti-CD20 or CD4 biologics and other cytokine inhibitors or biologics to T-cell or B-cell receptors or interleukins.

In certain embodiments, the at least one other therapeutically active agent is selected from broad-spectrum antibiotic, anti-MRSA therapy and a low dose steroid.

In certain embodiments, the at least one other therapeutically active agent is an antimicrobial agent or an antibiotic.

In certain embodiments, the at least one other therapeutically active agent is selected from an inhaled corticosteroid, a long acting beta agonist, a combination of an inhaled corticosteroid and a long acting beta agonist, a short acting beta agonist, a leukotriene modifier, an anti-IgE, a methylxanthine bronchodilator, a mast cell inhibitor, and a long-acting muscarinic antagonist.

In certain embodiments, the at least one other therapeutically active agent is selected from protein tyrosine kinase inhibitor, a CRTH2/D-prostanoid receptor antagonist, an epinephrine inhalation aerosol, and a combination of a phosphodiesterase-3 inhibitor and a phosphodiesterase-4 inhibitor.

In certain embodiments, the at least one other therapeutically active agent is selected from a long acting beta agonist, a long-acting inhaled anticholinergic or muscarinic antagonist, a phosphodiesterase inhibitor, a combination an inhaled corticosteroid long acting beta agonist, a short acting beta agonist, and an inhaled corticosteroid.

In certain embodiments, the at least one other therapeutically active agent is an antimycobacterial agent or a bactericidal antibiotic.

In certain embodiments, the at least one other therapeutically active agent is selected from an oral corticosteroid, anti-thymocyte globulin, thalidomide, chlorambucil, a calcium channel blocker, a topical emollient, an ACE inhibitor, a serotonin reuptake inhibitor, an endothelin-I receptor inhibitor, an anti-fibrotic agent, a proton-pump.

In certain embodiments, the at least one other therapeutically active agent is selected from a cystic fibrosis transmembrane conductance regulator potentiator, a mucolytic agent, pancreatic enzymes, a bronchodilator, an antibiotic, or ivacftor/lumacaftor, ataluren, and tiopropium bromide.

In certain embodiments, the at least one other therapeutically active agent is a ciliary neurotrophic growth factor or a gene transfer agent.

In certain embodiments, the at least one other therapeutically active agent is selected from ophthalmic intravitreal injections, an anti-vascular endothelial growth factor inhibitor, and a ciliary neurotrophic growth factor agent.

In certain embodiments, the at least one other therapeutically active agent is selected from a trivalent (IIV3) inactivated influenza vaccine, a quadrivalent (IIV4) inactivated influenza vaccine, a trivalent recombinant influenza vaccine, a quadrivalent live attenuated influenza vaccine, an antiviral agent, or inactivated influenza vaccine.

In certain embodiments, the at least one other therapeutically active agent is selected from a monoclonal antibody, a polyclonal anti-T-cell antibody, an anti-thymocyte gamma globulin-equine antibody, an antithymocyte globulin-rabbit antibody, an anti-CD40 antagonist, a JAK inhibitor, and an anti-TCR murine mAb.

In certain embodiments, the at least one other therapeutically active agent is selected from a topical immunomodulator or calcineurin inhibitor, a topical corticosteroid, an oral corticosteroid, an interferon gamma, an antihistamine, or an antibiotic.

### Dosing

The phrases “parenteral administration” and “administered parenterally” as used herein means modes of administration other than enteral and topical administration, usually by injection and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration”, “administered systemically”, “peripheral administration” and “administered peripherally” as used herein mean the administration of a compound, drug or other material other than directly into the central nervous system, such that it enters the patient's system and, thus, is subject to metabolism and other like processes, for example, subcutaneous administration. These compounds may be administered to humans and other animals for therapy by any suitable route of administration, including orally, nasally, as by, for example, a spray, rectally, intravaginally, parenterally, intracistemally and topically, as by powders, ointments or drops, including buccally and sublingually.

Regardless of the route of administration selected, the compounds of the present disclosure, which may be used in a suitable hydrated form and/or the pharmaceutical compositions of the present disclosure, are formulated into pharmaceutically acceptable dosage forms by conventional methods known to those of skill in the art.

Actual dosage levels of the active ingredients in the pharmaceutical compositions of this disclosure may be varied so as to obtain an amount of the active ingredient that is effective to achieve the desired therapeutic response for a particular patient, composition and mode of administration, without being toxic to the patient.

The selected dosage level will depend upon a variety of factors including the activity of a particular compound of the present disclosure employed or, salt or solvate thereof, the route of administration, the time of administration, the rate of excretion or metabolism of a particular compound being employed, the duration of the treatment, other drugs, compounds and/or materials used in combination with a particular compound employed, the age, sex, weight, condition, general health and prior medical history of the patient being treated and like factors well known in the medical arts. A daily, weekly or monthly dosage (or other time interval) can be used.

A physician or veterinarian having ordinary skill in the art can readily determine and prescribe the effective amount of the pharmaceutical composition required. For example, the physician or veterinarian could start doses of the compounds of the disclosure employed in the pharmaceutical composition at levels lower than that required to achieve the desired therapeutic effect and then gradually increasing the dosage until the desired effect is achieved.

In general, a suitable daily dose of a compound of the disclosure will be that amount of the compound that is the lowest dose effective to produce a therapeutic effect (e.g., inhibit necrosis). Such an effective dose will generally depend upon the factors described above.

Generally doses of the compounds of this disclosure for a patient, when used for the indicated effects, will range from about 0.0001 to about 100 mg per kg of body weight per day.

Preferably the daily dosage will range from 0.001 to 50 mg of compound per kg of body weight and even more preferably from 0.01 to 10 mg of compound per kg of body weight.

If desired, the effective daily dose of the active compound may be administered as two, three, four, five, six or more sub-doses administered separately at appropriate intervals throughout the day, optionally, in unit dosage forms. In certain embodiments, the present disclosure relates to compounds for inhibiting cell death, wherein the compounds are represented by formula (I). In certain embodiments, the compounds of the present disclosure are inhibitors of cell death. In any event, the compounds of the present disclosure preferably exert their effect on inhibiting cell death at a concentration less than about 50 micromolar, more preferably at a concentration less than about 10 micromolar and most preferably at a concentration less than 1 micromolar. The compounds of the disclosure can be tested in standard animal models of stroke and standard protocols such as described by Hara, H., et al. Proc. Natl. Acad. Sci. USA, 1997.94(5): 2007-12.

When the compounds of the present disclosure are administered as pharmaceuticals, to humans and animals, they can be given per se or as a pharmaceutical composition containing, for example, 0.1% to 99.5% (more preferably, 0.5% to 90%) of active ingredient in combination with a pharmaceutically acceptable carrier.

The compounds of the present disclosure or the compositions thereof may be administered once, twice, three or four times daily, using any suitable mode described above. Also, administration or treatment with the compounds may be continued for a number of days; for example, commonly treatment would continue for at least 7 days, 14 days or 28 days, for one cycle of treatment. Treatment cycles are well known and are frequently alternated with resting periods of about 1 to 28 days, commonly about 7 days or about 14 days, between cycles. The treatment cycles, in certain embodiments, may also be continuous.

When administered orally, the total daily dosage for a human subject may be between 1 mg and 1,000 mg, between about 1,000-2,000 mg/day, between about 10-500 mg/day, between about 50-300 mg/day, between about 75-200 mg/day or between about 100-150 mg/day.

The daily dosage may also be described as a total amount of a compound described herein administered per dose or per day. Daily dosage of a compound may be between about 1 mg and 4,000 mg, between about 2,000 to 4,000 mg/day, between about 1 to 2,000 mg/day, between about 1 to 1,000 mg/day, between about 10 to 500 mg/day, between about 20 to 500 mg/day, between about 50 to 300 mg/day, between about 75 to 200 mg/day or between about 15 to 150 mg/day. In certain embodiments, the method comprises administering to the subject an initial daily dose of about 1 to 800 mg of a compound described herein and increasing the dose by increments until clinical efficacy is achieved. Increments of about 5, 10, 25, 50 or 100 mg can be used to increase the dose. The dosage can be increased daily, every other day, twice per week or once per week.

In certain embodiments, a compound or pharmaceutical preparation is administered orally. In certain embodiments, the compound or pharmaceutical preparation is administered intravenously. Alternative routes of administration include sublingual, intramuscular and transdermal administrations.

The preparations of the present disclosure may be given orally, parenterally, topically or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. In certain embodiments, the administration is oral.

### EXAMPLES

### Abbreviations

| | |
|---|---|
| CMC | Carboxy-methyl-cellulose |
| DCM | Dichloromethane |
| DEA | Diethylamine |
| DIPEA | Diisopropylethylamine |
| DMF | Dimethylformamide |
| DMSO | Dimethylsulfoxide |
| Eq. | equivalent |
| ES | Electro spray |
| EtOAc | Ethyl acetate |
| HATU | 1-[Bis(dimethylamino)methylene]- |
| | 1H-1,2,3-triazolo[4,5-b]pyridinium |
| | 3-oxide hexafluorophosphate |
| HBTU | |
| HPLC | High pressure liquid chromatography |
| IL6 | Interleukin 6 |
| LC/MS | Liquid chromatography mass spectrum |
| m/z | Mass per charge |
| MeOH | Methanol |
| MHz | Mega Hertz |
| min | Minute(s) |
| N | Normal |
| NMR | Nuclear magnetic resonance |
| PBS | Phosphate buffered saline |
| PE | Petrol ether |
| TFA | Trifluoroacetic acid |
| THF | Tetrahydrofurane |
| TLC | Thin layer chromatography |
| TNF | Tumor Necrosis Factor |
| rH | Relative humidity |
| tR | retention time |
| UV | ultraviolet |

### NMR

400 MHz: NMR spectra were recorded on a Bruker AVANCE II 400 spectrometer operating at a proton frequency of 400.23 MHz. The instrument was equipped with a 5 mm BBI room temperature probe head. Alternatively, a Bruker AVANCE III HD 400 MHz, or a Bruker AVANCE NEO 400 MHz was used.

### Analytical LC/MS Equipment for Method A

For retention time and mass detection a LC/MS-system from Agilent (LC 1200 Series/MS 6120 quadrupole LC/MS, LC 1260 infinity/MS 6120 quadrupole LC/MS or LC 1260 Infinity II/MSD Infinity Lab) was used. Molecular weights are given in gram per mol [g/mol], detected masses in mass per charge [m/z].

### Analytical LC/MS Equipment for Method B

Retention time and mass detection were done on a Waters Acquity UHPLC system coupled with a Waters SQD mass detector. The injection volume was 1.0 μl. Molecular weights are given in gram per mol [g/mol], detected masses in mass per charge [m/z].

### LC/MS-Method A

Gradient: 98% H₂O (0.05% formic acid)/2% acetonitrile (0.035% formic acid) for 0.2 min, then from 98% H₂O (0.05% formic acid) to 98% acetonitrile (0.035% formic acid) in 3.6 min, then 98% acetonitrile (0.035% formic acid) for 0.5 min, flow rate: 1.0 ml/min, column: 2.1×50 mm Waters ACQUITY UPLC BEH C18, 1.7 μm, 55° C.
UV data: retention time ad λ=220 nm given in min
MS data: ES+ ionisation, m/z given as [M+H]⁺ unless otherwise noted

### LC/MS-Method B

Gradient: From 95% H₂O (0.0375% TFA)/5% acetonitrile (0.01875% TFA) to 5% H₂O (0.0375% TFA)/95% acetonitrile (0.01875% TFA) in 0.8 min, flow rate: 1.5 ml/min, column:
Kinetex EVO C18 2.1×30 mm, 5 μm, 50° C.

UV data: retention time ad λ 220 nm given in min
MS data: ES+ ionisation, m/z given as [M+H]⁺ unless otherwise noted

### Analytical Chiral HPLC

SFC: SHIMADZU LC-30AD sf system
LC: Agilent 1100 series system

### Salts

In compounds described as HCl-, TFA- or as another salt the exact amount of the respective salt is usually not determined. Therefore, the amount of the salt can range from as low as 0.01 eq. up to 5.0 eq. depending on the chemical structure (e.g. number of basic centres).

### Chiral Purity

Compounds are drawn and named as a single enantiomer, if the enantiomeric ratio exceeded 90:10. For enantiomeric ratios below 90:10 the racemic form is used.

### Synthetic Route A:

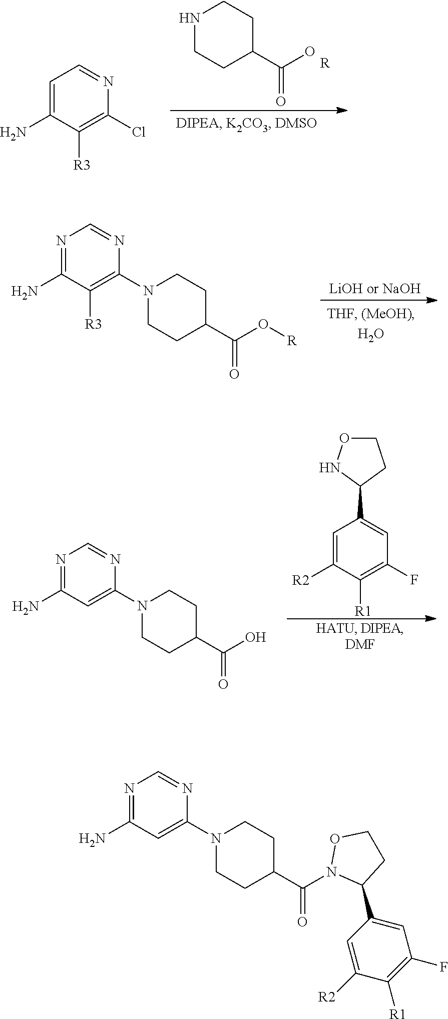

### Synthetic Route B:

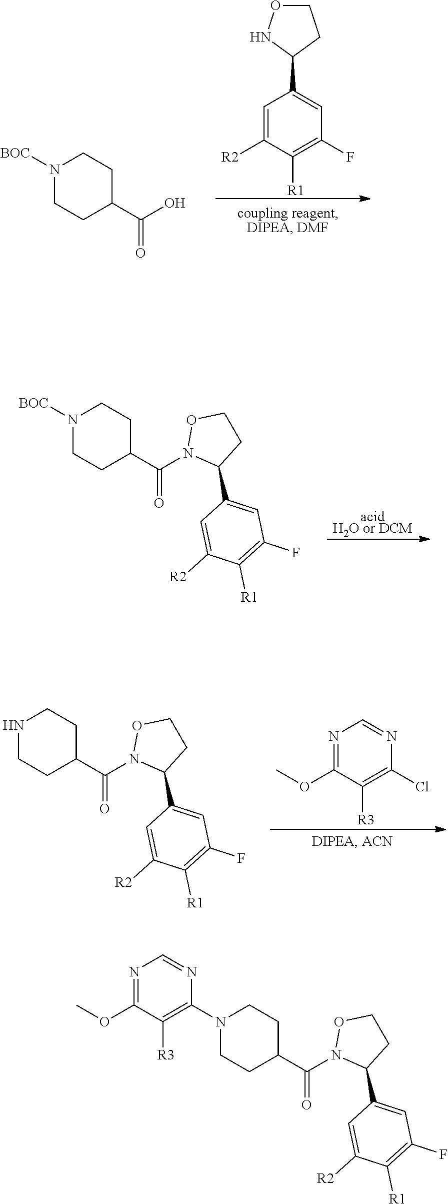

Examples according to synthetic route A:

### Example 1

### [1-(6-aminopyrimidin-4-yl)-4-piperidyl]-[(3S)-3-(3,5-difluorophenyl)isoxazolidin-2-yl]methanone

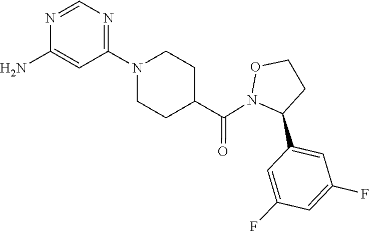 [SMILES: Nc1cc(N2CCC(C(=O)N3OCC[C@H]3c3cc(F)cc(F)c3)CC2)ncn1]

### Step 1:

### Methyl 1-(6-aminopyrimidin-4-yl)piperidine-4-carboxylate

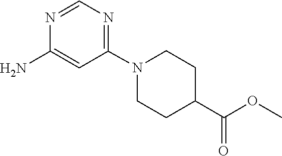 [SMILES: COC(=O)C1CCN(c2cc(N)ncn2)CC1]

To a solution of 6-chloropyrimidin-4-amine (2 g, 15.44 mmol, 1 eq.) and methyl piperidine-4-carboxylate (3.32 g, 23.16 mmol, 1.5 eq.) in DMSO (20 mL) was added DIPEA (9.98 g, 77.20 mmol, 13.45 mL, 5 eq.). The mixture was stirred at 80° C. for 12 hours. Another amount of methyl piperidine-4-carboxylate (2.21 g, 15.44 mmol, 1 eq.) and K₂CO₃ (4.27 g, 30.88 mmol, 2 eq.) was added and again the mixture was stirred at 80° C. for 12 hours. The reaction mixture was filtered, diluted with water (100 mL) and extracted with EtOAc (80 mL*3). The combined organic layers were washed with brine (200 mL), dried over Na₂SO₄, filtered and concentrated. The crude product was triturated with PE/EtOAc (2:1, 45 mL) at 25° C. for 30 min to give the title compound (3.05 g, 12.39 mmol, 80.3% yield) as a yellow solid.

LC/MS: m/z=237.1 [M+H]⁺; tR: 0.32 min (LC/MS method A).

¹H NMR: (400 MHz, CDCl₃): δ ppm 8.18 (d, J=0.6 Hz, 1H), 5.60 (d, J=0.9 Hz, 1H), 4.57 (br s, 2H), 4.20 (td, J=3.6, 13.5 Hz, 2H), 3.70 (s, 3H), 2.99 (ddd, J=2.9, 11.1, 13.6 Hz, 2H), 2.58 (tt, J=4.2, 11.0 Hz, 1H), 2.00-1.94 (m, 2H), 1.77-1.67 (m, 2H)

### Step 2:

### 1-(6-Aminopyrimidin-4-yl)piperidine-4-carboxylic acid

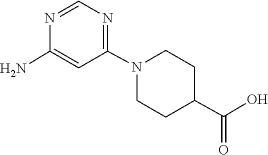 [SMILES: Nc1cc(N2CCC(C(=O)O)CC2)ncn1]

To a solution of methyl 1-(6-aminopyrimidin-4-yl)piperidine-4-carboxylate (500 mg, 2.12 mmol, 1 eq.) in THF (5 mL) was added LiOH*H₂O (1 M, 4.23 mL, 2 eq.). The mixture was stirred at 25° C. for 1 hour. The reaction mixture was adjusted to pH=5 with 1 N HCl aqueous solution, then filtered and the filter cake was dried to give the title compound (353 mg, crude) as an off white solid.

¹H NMR: (400 MHz, DMSO-d₆): δ ppm 13.06-11.40 (m, 1H), 7.94 (s, 1H), 6.16 (s, 2H), 5.61 (s, 1H), 4.06 (br d, J=13.2 Hz, 2H), 3.00-2.83 (m, 2H), 2.49-2.44 (m, 1H), 1.82 (br dd, J=2.9, 13.2 Hz, 2H), 1.51-1.40 (m, 2H)

### Step 3:

### [1-(6-aminopyrimidin-4-yl)-4-piperidyl]-[(3S)-3-(3,5-difluorophenyl)isoxazolidin-2-yl]methanone

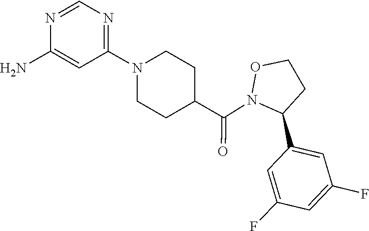 [SMILES: Nc1cc(N2CCC(C(=O)N3OCC[C@H]3c3cc(F)cc(F)c3)CC2)ncn1]

To a solution of 1-(6-aminopyrimidin-4-yl)piperidine-4-carboxylic acid (46.7 mg, 0.210 mmol, 1.05 eq.) in DMF (1 mL) was added HATU (114 mg, 0.300 mmol, 1.5 eq.), DIPEA (64.6 mg, 0.500 mmol, 87 uL, 2.5 eq.), and (3S)-3-(3,5-difluorophenyl)isoxazolidine (37.0 mg, 0.200 mmol, 1.00 eq.) and the mixture was stirred at 25° C. for 4.5 hours. The reaction mixture was purified by prep-HPLC (column: Agilent Prep-C18 5 μm 30×100 mm; eluents: water and acetonitrile; flow: 50 ml/min; gradient: 1 min 10% acetonitrile, in 12 min from 10 to 100% acetonitrile, 2 min 100% acetonitrile) to give 29 mg (74.0 μmol, 37.0% yield) of the title compound.

LC/MS: m/z=390.2 [M+H]⁺; tR: 1.32 min (LC/MS method A)

¹H NMR: (400 MHz, DMSO-d₆): δ ppm 7.94 (s, 1H), 7.13 (tt, J=9.34, 9.34, 2.28, 2.28 Hz, 1H), 6.99 (m, 2H), 6.14 (s, 2H), 5.62 (s, 1H), 5.35 (m, 1H), 4.22 (m, 3H), 3.90 (m, 1H), 3.02 (br s, 1H), 2.89 (m, 3H), 2.20 (m, 1H), 1.88 (br d, J=11.49 Hz, 1H), 1.68 (br d, J=12.72 Hz, 1H), 1.47 (m, 2H)

### Example 2

### 5-[(3S)-2-[1-(6-aminopyrimidin-4-yl)piperidine-4-carbonyl]isoxazolidin-3-yl]-3-fluoro-2-methyl-benzonitrile

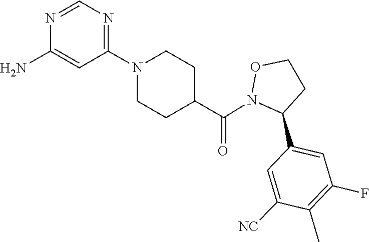 [SMILES: Cc1c(F)cc([C@@H]2CCON2C(=O)C2CCN(c3cc(N)ncn3)CC2)cc1C#N]

This compound has been synthesized as described for example 1 to yield 49 mg (60%).

LC/MS: m/z=411.2 [M+H]⁺; tR: 1.34 min (LC/MS method A)

¹H NMR: (400 MHz, DMSO-d₆): δ ppm 7.94 (s, 1H), 7.55 (s, 1H), 7.43 (d, J=10.39 Hz, 1H), 6.13 (s, 2H), 5.61 (s, 1H), 5.34 (m, 1H), 4.28 (m, 1H), 4.18 (br t, J=11.49, 11.49 Hz, 2H), 3.92 (m, 1H), 3.00 (m, 1H), 2.88 (m, 3H), 2.37 (d, J=1.71 Hz, 3H), 2.23 (m, 1H), 1.87 (br d, J=11.62 Hz, 1H), 1.69 (br d, J=12.72 Hz, 1H), 1.47 (m, 2H)

### Example 3

### 3-[(3S)-2-[1-(6-Aminopyrimidin-4-yl)piperidine-4-carbonyl]isoxazolidin-3-yl]-5-fluoro-benzonitrile

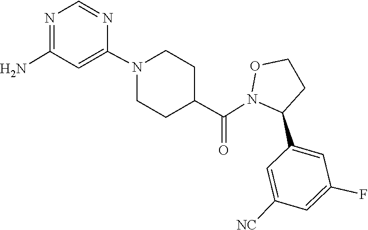 [SMILES: N#Cc1cc(F)cc([C@@H]2CCON2C(=O)C2CCN(c3cc(N)ncn3)CC2)c1]

This compound has been synthesized as described for Example 1 to yield 200 mg (37%) of the title compound.

LC/MS: m/z=397.2 [M+H]⁺; tR: 0.77 min (LC/MS method B).

¹H NMR: (400 MHz, DMSO-d₆): δ ppm 7.94 (s, 1H), 7.81-7.72 (m, 1H), 7.61 (s, 1H), 7.48 (br d, J=9.5 Hz, 1H), 6.16 (s, 2H), 5.61 (s, 1H), 5.38 (br dd, J=6.7, 8.2 Hz, 1H), 4.28 (dt, J=2.9, 7.7 Hz, 1H), 4.18 (br dd, J=10.1, 12.2 Hz, 2H), 3.96-3.88 (m, 1H), 3.02 (br t, J=10.9 Hz, 1H), 2.94-2.82 (m, 3H), 2.29-2.18 (m, 1H), 1.88 (br d, J=11.5 Hz, 1H), 1.69 (br d, J=12.6 Hz, 1H), 1.54-1.40 (m, 2H)

SFC: Column: Chiralcel OJ-3 50×4.6 mm I.D., 3 μm, eluent: MeOH (0.05% DEA) and CO₂;

gradient: 5-40% MeOH in CO₂; flow rate: 3 mL/min; column temperature: 35° C., 100 bar): tR: 1.71 min (100%)

Examples according to synthetic route B:

### Example 4

### (S)-3-fluoro-5-(2-(1-(6-methoxypyrimidin-4-yl)piperidine-4-carbonyl)isoxazolidin-3-yl)benzonitrile

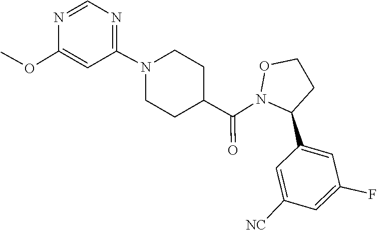 [SMILES: COc1cc(N2CCC(C(=O)N3OCC[C@H]3c3cc(F)cc(C#N)c3)CC2)ncn1]

### Step 1:

### tert-butyl 4-[(3S)-3-(3-cyano-5-fluoro-phenyl)isoxazolidine-2-carbonyl]piperidine-1-carboxylate

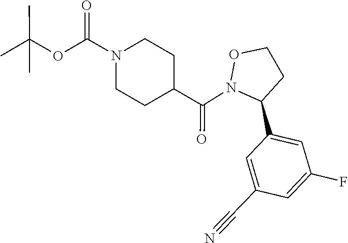 [SMILES: CC(C)(C)OC(=O)N1CCC(C(=O)N2OCC[C@H]2c2cc(F)cc(C#N)c2)CC1]

A solution of (S)-3-fluoro-5-(isoxazolidin-3-yl)benzonitrile 2,2,2-trifluoroacetate (500 mg, 1.63 mmol), 1-(tert-butoxycarbonyl)piperidine-4-carboxylic acid (411.8 mg, 1.80 mmol), HATU (1.24 g, 3.27 mmol) and DIPEA (627 μl, 3.59 mmol) in DMF (5 ml) was stirred for 16 hrs. The reaction mixture was diluted with saturated NaHCO₃ solution and water and extracted with ethyl acetate (3 times). The combined organic layers were dried with Na2SO3, filtered and evaporated. The crude product was purified via prep. HPLC (column: Agilent Prep-C18 10 μm 30×100 mm; eluents: water and acetonitrile; flow: 50 ml/min; gradient: 1 min 10% acetonitrile, in 12 min from 10 to 100% acetonitrile, 2 min 100% acetonitrile) to give 410 mg (62%) of the title compound.

LC/MS: m/z=348.34 [M+H-isobuten]⁺; tR: 2.28 min (LC/MS method A)

¹H NMR: (400 MHz, DMSO-d₆): δ ppm 7.78 (m, 1H), 7.61 (s, 1H), 7.48 (br d, J=9.54 Hz, 1H), 5.38 (m, 1H), 4.27 (m, 1H), 3.91 (m, 3H), 2.89 (m, 4H), 2.22 (m, 1H), 1.84 (br d, J=11.49 Hz, 1H), 1.65 (br d, J=12.10 Hz, 1H), 1.39 (m, 9H)

### Step 2:

### 3-fluoro-5-[(3S)-2-(piperidine-4-carbonyl)isoxazolidin-3-yl]benzonitrile

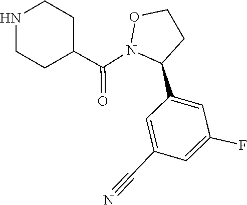 [SMILES: N#Cc1cc(F)cc([C@@H]2CCON2C(=O)C2CCNCC2)c1]

A solution of tert-butyl (S)-4-(3-(3-cyano-5-fluorophenyl)isoxazolidine-2-carbonyl)piperidine-1-carboxylate (1.5 g, 3.72 mmol) and trifluoroacetic acid (3 ml, 38.94 mmol) in dichloromethane (30 ml) was stirred at room temperature for 16 hrs. The reaction mixture was evaporated and lyophilized two times to give 1.65 g (98%) of the title compound as trifluoroacetic acid salt, which was used in the next step without further purification.

LC/MS: m/z=304.2 [M+H]⁺; tR: 0.92 min (LC/MS method A)

### Step 3:

### (S)-3-fluoro-5-(2-(1-(6-methoxypyrimidin-4-yl)piperidine-4-carbonyl)isoxazolidin-3-yl)benzonitrile

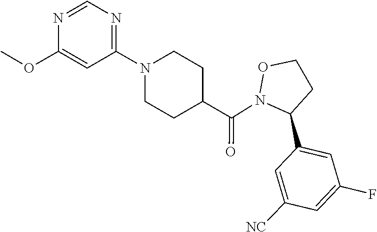 [SMILES: COc1cc(N2CCC(C(=O)N3OCC[C@H]3c3cc(F)cc(C#N)c3)CC2)ncn1]

To a suspension of (S)-3-fluoro-5-(2-(piperidine-4-carbonyl)isoxazolidin-3-yl)benzonitrile 2,2,2-trifluoroacetate (50 mg, 110.73 μmol), and 4-chloro-6-methoxypyrimidine (24.0 mg, 166.1 μmol) in acetonitrile (1.5 mL) DIPEA (96.7 μl, 553.6 μmol) was added. The reaction mixture was heated to 120° C. under microwave conditions for 1 hour. The reaction mixture was purified by revered phase chromatography (column: Agilent Prep-C18 10 μm 30×100 mm; eluents: H₂O and acetonitrile, flow: 50 ml/min; gradient: 1 min 10% acetonitrile, in 12 min from 10 to 100% acetonitrile, 2 min at 100% acetonitrile) to yield 22 mg (48%) of the title compound.

LC/MS: m/z=412.3 [M+H]⁺; tR: 1.73 min (LC/MS method A)

¹H NMR: (400 MHz, DMSO-d₆): δ ppm 8.23 (s, 1H), 7.77 (m, 1H), 7.61 (s, 1H), 7.48 (br d, J=9.54 Hz, 1H), 6.08 (s, 1H), 5.39 (m, 1H), 4.30 (m, 3H), 3.93 (m, 1H), 3.81 (s, 3H), 3.07 (br s, 1H), 3.03 (br d, J=2.93 Hz, 1H), 2.93 (m, 2H), 2.25 (m, 1H), 1.91 (br d, J=11.74 Hz, 1H), 1.72 (br d, J=12.96 Hz, 1H), 1.48 (m, 2H)

### Example 5

### 3-fluoro-5-[(3S)-2-[1-(5-fluoro-6-methoxy-pyrimidin-4-yl)piperidine-4-carbonyl]isoxazolidin-3-yl]benzonitrile

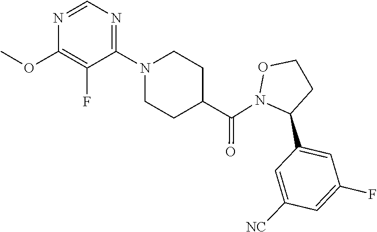 [SMILES: COc1ncnc(N2CCC(C(=O)N3OCC[C@H]3c3cc(F)cc(C#N)c3)CC2)c1F]

One method for synthesizing Example 5 is analog to the one described for example 4.

Example 5 was synthesized as byproduct of another synthesis as described below:

### Step 1:

### ethyl 1-(6-chloro-5-fluoro-pyrimidin-4-yl)piperidine-4-carboxylate

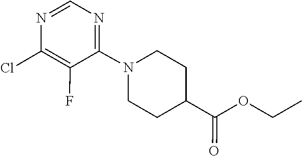 [SMILES: CCOC(=O)C1CCN(c2ncnc(Cl)c2F)CC1]

To a mixture of 4,6-dichloro-5-fluoro-pyrimidine (1.06 g, 6.36 mmol, 1 eq.) and ethyl piperidine-4-carboxylate (1.00 g, 0.98 ml, 6.36 mmol, 1 eq.) in acetonitrile (20 mL) DIPEA (3.288 g, 4.43 mL, 25.44 mmol, 4 eq.) was added. The reaction mixture was stirred at room temperature for 16 hours, diluted with ethyl acetate and extracted two times with 0.1 N HCl. The organic layer was washed with water, dried with Na₂SO₄ and lyophilized to give 1.65 g (90%) of the title compound, which was used without further purification in the next step.

LC/MS: m/z=288.1 [M+H]⁺; tR: 2.14 min (LC/MS method A)

### Step 2:

### ethyl 1-[5-fluoro-6-(4-methylpyrazol-1-yl)pyrimidin-4-yl]piperidine-4-carboxylate

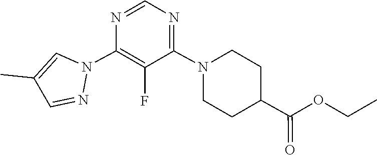 [SMILES: CCOC(=O)C1CCN(c2ncnc(-n3cc(C)cn3)c2F)CC1]

A mixture of ethyl 1-(6-chloro-5-fluoro-pyrimidin-4-yl)piperidine-4-carboxylate (300 mg, 1.04 mmol, 1 eq.), 4-methyl-1H-pyrazole (171 mg, 0.168 mL, 2.08 mmol, 2 eq.), potassium carbonate (576 mg, 4.17 mmol, 4 eq.) and copper(I)iodide (99 mg, 0.52 mmol, 0.5 eq.) in DMSO (1.5 mL) was stirred at 120° C. under microwave irradiation for 4 hours. The reaction mixture diluted with ethyl acetate, filtered and extracted with water. The organic layer dried with Na₂SO₄ and evaporated to give 294 mg (72%) of the title compound, which was used without further purification in the next step.

### Step 3:

### 1-(5-fluoro-6-methoxy-pyrimidin-4-yl)piperidine-4-carboxylic acid

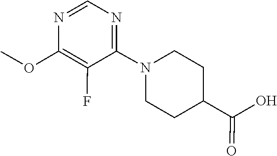 [SMILES: COc1ncnc(N2CCC(C(=O)O)CC2)c1F]

A solution of ethyl 1-[5-fluoro-6-(4-methylpyrazol-1-yl)pyrimidin-4-yl]piperidine-4-carboxylate (294 mg, 0.75 mmol, 1 eq.) in NaOH (2N in THF/MeOH 1:1:1) (5.7 mL, 3.75 mmol, 5 eq.) was stirred at 25° C. for 16 hours. The reaction mixture was acidified with 1 N H₂SO₄, extracted three times with ethyl acetate, dried with Na₂SO₄, filtered and evaporated. The residue was purified by reversed phase chromatography (column: YMC-Actus Triart prep. C18-S 10 μm, 250×30 mm, eluent: acetonitrile and water (0.05% TFA), flow 70 ml/min; gradient: 2 min 5% acetonitrile, in 24 min from 5 to 100% acetonitrile, 4 min 100% acetonitrile to yield 156 mg (68%) of expected product 1-[5-fluoro-6-(4-methylpyrazol-1-yl)pyrimidin-4-yl]piperidine-4-carboxylic acid and 32 mg (13%) of the title compound as byproduct.

LC/MS: m/z=256.1 [M+H]⁺; tR: 1.48 min (LC/MS method A)

¹H NMR: (400 MHz, DMSO-d₆): δ ppm 12.25 (br s, 1H), 8.07 (s, 1H), 4.20 (m, 2H), 3.91 (s, 3H), 3.13 (m, 2H), 2.58 (m, 1H), 1.88 (m, 2H), 1.56 (m, 2H)

### Step 4:

### 3-fluoro-5-[(3S)-2-[1-(5-fluoro-6-methoxy-pyrimidin-4-yl)piperidine-4-carbonyl]isoxazolidin-3-yl]benzonitrile

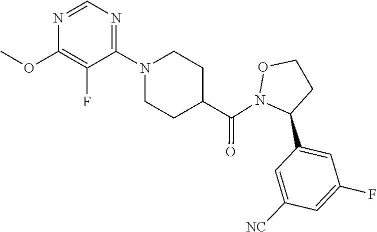 [SMILES: COc1ncnc(N2CCC(C(=O)N3OCC[C@H]3c3cc(F)cc(C#N)c3)CC2)c1F]

This compound was synthesized as described in example 1 step 3 to yield 11 mg (26%).

LC/MS: m/z=430.2 [M+H]⁺; tR: 2.23 min (LC/MS method A)

¹H NMR: (400 MHz, DMSO-d₆): δ ppm 8.07 (s, 1H), 7.77 (br d, J=7.46 Hz, 1H), 7.61 (s, 1H), 7.48 (br d, J=9.66 Hz, 1H), 5.38 (m, 1H), 4.29 (m, 2H), 3.91 (s, 3H), 3.13 (q, J=11.62, 11.62, 11.62 Hz, 3H), 2.90 (m, 1H), 2.33 (m, 1H), 2.23 (m, 1H), 1.94 (br d, J=13.94 Hz, 1H), 1.73 (br s, 1H), 1.58 (br d, J=12.59 Hz, 2H), 1.24 (s, 2H)

### Example 6

### 3-fluoro-5-[(3S)-2-[1-(6-methoxypyrimidin-4-yl)piperidine-4-carbonyl]isoxazolidin-3-yl]-2-methyl-benzonitrile

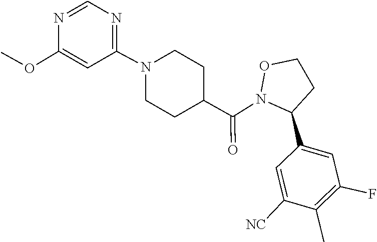 [SMILES: COc1cc(N2CCC(C(=O)N3OCC[C@H]3c3cc(F)c(C)c(C#N)c3)CC2)ncn1]

This compound has been synthesized as described for Example 1 to yield 42 mg (50% yield).

LC/MS: m/z=426.2 [M+H]⁺; tR: 1.89 min (LC/MS method A).

¹H NMR: (400 MHz, DMSO-d₆): δ ppm 8.23 (s, 1H), 7.55 (s, 1H), 7.44 (s, 1H), 7.41 (m, 1H), 6.09 (s, 1H), 5.34 (m, 1H), 4.29 (m, 2H), 3.92 (m, 1H), 3.82 (s, 3H), 3.01 (m, 3H), 2.88 (m, 1H), 2.37 (d, J=1.83 Hz, 3H), 2.22 (m, 1H), 1.90 (br d, J=11.25 Hz, 1H), 1.71 (br d, J=11.86 Hz, 1H), 1.47 (m, 2H)

### Example 7

### [(3S)-3-(3-chloro-5-fluoro-phenyl)isoxazolidin-2-yl]-[1-(6-methoxypyrimidin-4-yl)-4-piperidyl]methanone

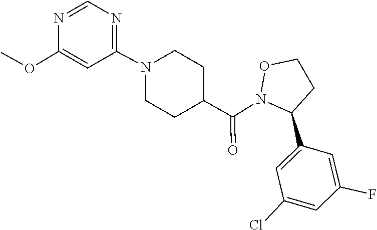 [SMILES: COc1cc(N2CCC(C(=O)N3OCC[C@H]3c3cc(F)cc(Cl)c3)CC2)ncn1]

This compound has been synthesized as described for example 1 to yield 30 mg (36% yield).

LC/MS: m/z=421.1 [M+H]⁺; tR: 2.04 min (LC/MS method A)

¹H NMR: (400 MHz, DMSO-d₆): δ ppm 8.23 (d, J=0.61 Hz, 1H), 7.33 (dt, J=8.68, 2.14, 2.14 Hz, 1H), 7.19 (s, 1H), 7.10 (br d, J=9.54 Hz, 1H), 6.08 (s, 1H), 5.34 (m, 1H), 4.29 (m, 3H), 3.90 (m, 1H), 3.81 (s, 3H), 3.01 (m, 3H), 2.89 (m, 1H), 2.21 (m, 1H), 1.90 (br d, J=12.84 Hz, 1H), 1.71 (br d, J=11.25 Hz, 1H), 1.48 (m, 2H)

### Evaluation of Receptor-Interacting Protein Kinase 1 Inhibition.

The catalytic activity of RIPK1 was measured by monitoring the conversion of Adenosine triphosphate (ATP) to Adenosine diphosphate (ADP) due to autophosphorylation using an ADP-Glo kinase kit (Promega, catalog no. V9104).

2 μl recombinantly produced hRIPK1 (aa 1-375) fusion protein (end concentration 3.6 μg/ml) and 2 μl compound (end concentration 33300-1.69 nM; DMSO end concentration 1%) were incubated for 30 minutes at room temperature and then 2 μl ATP (ADP Glo kit, end concentration 50 μM) were added. After another 240 minutes incubation at room temperature, 5 μl Promega ADP-Glo reagent I were added to quench the reaction and deplete unconsumed ATP. After an incubation period of 30 minutes, 10 μl Promega ADP-Glo detection reagent II were added resulting in conversion of ADP to ATP, which generates a light-reaction between luciferase and luciferin. Luminescence was quantified after 30 minutes with a Pherastar FS (BMG LABTECH, Ortenberg).

For the dose response experiments an IC₅₀ value with 95% confidence interval was calculated using the 4-parameter logistic model according to Ratkowsky and Reedy with constraints for lower and upper asymptotes at 0% and 100%. The adjustment was obtained by nonlinear regression using the Levenberg Marquardt algorithm.

### Cellular Assay in U937 Cells to Measure the Activity of RIPK1-Inhibitors on Cell Death (Necroptosis).

Upon TNF-Receptor I ligation, Ser/Thr kinase RIPK1 is recruited to a transient receptor complex I. Upon modification of RIPK1 which promotes activation of RIPK1, complex IIb can form, that involves recruitment of RIPK3 and MLKL (mixed lineage-kinase domain-like protein) which then translocates from the cytosol to the plasma membrane to execute cell death (Cai, Z. et al, Nat. Cell Biol. (2014)16:55-65).

Cell death was quantified in 96 well plates by determination of the amount of live cell using a CellTiter 96 AQueous reagent (Promega), a calorimetric method to measure the amount of live cells by reducing tetrazolium compound [3-(4,5-dimethylthiazol-2-yl)-5-(3-carboxymethoxyphenyl)-2-(4-sulfophenyl)-2H-tetrazolium, inner salt; MTS] into formazan. Absorbance of formazan was read at 490 nm. The inhibitory activity of the test compound was quantified in a concentration response curve (CRC) experiment.

Compounds were obtained as 10 mM stock solutions and were diluted 1 to 10 volumes with DMSO to yield a 1 mM solution. From this solution 2 μl were diluted with 998 μl growth medium. 100 μl of 2 μM compound solution was further diluted sequentially with a dilution factor of 2.5 by adding 150 μl growth medium. A total of 10 concentrations were tested ranging from 10 μM to 0.26 nM or from 1 μM to 0.07 nM.

U937 cells were cultured in RPMI1640 Glutamax and 10% heat inactivated FBS. 50 μl cell suspension containing 1×106 cells/ml supplemented with 50 μM zVAD.fink (Benzyloxycarbonyl-Val-Ala-Asp (OMe) fluoromethylketone) and 100 ng/ml recombinant human TNFa were dispensed in each well of a 96-well plate. 50 μl of compound dilutions (see above) were added and the cell suspension incubated overnight (18 to 24 hrs) at 37° C., 5% CO₂ in a humidified atmosphere (95% rH). High (no compound) and low control (no TNFa, zVAD.fink) were tested with 7 replicates; all compound concentrations were tested in duplicates on each experimental plate.

CellTiter 96 Aqueous reagent was mixed (100 μl PMS (phenazine methosulfate) solution/2 ml MTS (3-(4,5-dimethyldiazol-2-yl)-5-(3-carboxymethoxyphenyl)-2-(4-sulfophenyl)-2H-tetrazolium, inner salt) solution) and 20 μl were added per well. After 4 hrs incubation at 37° C. (5% CO₂ 95% rH) optical density was measured at 490 nm on a microplate reader (Tecan Infinite M1000).

The % inhibition is expressed as percentage of the maximal inhibition value obtained in the absence of TNFa/zVAD.fnc. For each dose response experiment an IC₅₀ value with 95% confidence interval was calculated using the 4-parameter logistic model according to Ratkowsky and Reedy without constraints using an internal application (Biost@t-Speed LTS V2.3).

TABLE 2
activity data of all examples

| | ADP Glo assay | U937 cell assay |
|---|---|---|
| Example | IC₅₀ [nM] | IC₅₀ [nM] |
| 1 | 13 | |
| 2 | 23 | |
| 3 | 17 | 22 |
| 4 | 34 | 80 |
| 5 | 18 | 8 |
| 6 | 22 | |
| 7 | 17 | |

1. A compound of formula I: 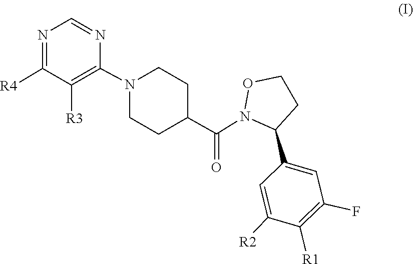
wherein
R1 is H or C₁-C₄ allyl,
R2 is CN or halogen,
R3 is H or halogen,
R4 is NH₂ or OCH₃,
or a pharmaceutically acceptable salt, solvate, or stereoisomer thereof,
wherein the compound of formula I with R1═H, R2=F, R3=H and R4=OCH₃ is excluded.

2. A compound of formula I according to claim 1,
wherein
R4 is NH₂,
or a pharmaceutically acceptable salt, solvate, or stereoisomer thereof.

3. A compound of formula I according to claim 1,
wherein
R4 is OCH₃,
or a pharmaceutically acceptable salt, solvate, or stereoisomer thereof.

4. A compound of formula I according to any one of claims 1, 2 or 3,
wherein
R3 represents F,
or a pharmaceutically acceptable salt, solvate, or stereoisomer thereof.

5. A compound of formula I according to claim 1 or 2,
wherein
when R2 is halogen, then R4 is NH₂,
or a pharmaceutically acceptable salt, solvate, or stereoisomer thereof.

6. A compound of formula I according to claim 1 to 4,
wherein
R2 is CN,
or a pharmaceutically acceptable salt, solvate, or stereoisomer thereof.

7. A compound of formula I according to claim 1 to 3 or 6,
wherein
R1 represents H,
R2 represents CN,
R3 represents H;
R4 represents NH₂ or OCH₃,
or a pharmaceutically acceptable salt, solvate, or stereoisomer thereof.

8. A compound of formula I according to claim 1, selected from
3-[(3S)-2-[1-(6-Aminopyrimidin-4-yl)piperidine-4-carbonyl]isoxazolidin-3-yl]-5-fluoro-benzonitrile,
[1-(6-aminopyrimidin-4-yl)-4-piperidyl]-[(3S)-3-(3,5-difluorophenyl)isoxazolidin-2-yl]methanone,
5-[(3S)-2-[1-(6-aminopyrimidin-4-yl)piperidine-4-carbonyl]isoxazolidin-3-yl]-3-fluoro-2-methyl-benzonitrile,
(S)-3-fluoro-5-(2-(1-(6-methoxypyrimidin-4-yl)piperidine-4-carbonyl)isoxazolidin-3-yl)benzonitrile
3-fluoro-5-[(3S)-2-[1-(5-fluoro-6-methoxy-pyrimidin-4-yl)piperidine-4-carbonyl]isoxazolidin-3-yl]benzonitrile,
3-fluoro-5-[(3S)-2-[1-(6-methoxypyrimidin-4-yl)piperidine-4-carbonyl]isoxazolidin-3-yl]-2-methyl-benzonitrile and
[(3S)-3-(3-chloro-5-fluoro-phenyl)isoxazolidin-2-yl]-[1-(6-methoxypyrimidin-4-yl)-4-piperidyl]methanone,
or a pharmaceutically acceptable salt, solvate, or stereoisomer thereof.

9. A compound of formula I according to claim 1, selected from
3-[(3S)-2-[1-(6-Aminopyrimidin-4-yl)piperidine-4-carbonyl]isoxazolidin-3-yl]-5-fluoro-benzonitrile,
[1-(6-aminopyrimidin-4-yl)-4-piperidyl]-[(3S)-3-(3,5-difluorophenyl)isoxazolidin-2-yl]methanone and
5-[(3S)-2-[1-(6-aminopyrimidin-4-yl)piperidine-4-carbonyl]isoxazolidin-3-yl]-3-fluoro-2-methyl-benzonitrile,
or a pharmaceutically acceptable salt, solvate, or stereoisomer thereof.

10. A compound of formula I according to claim 1, selected from
(S)-3-fluoro-5-(2-(1-(6-methoxypyrimidin-4-yl)piperidine-4-carbonyl)isoxazolidin-3-yl)benzonitrile
3-fluoro-5-[(3S)-2-[1-(5-fluoro-6-methoxy-pyrimidin-4-yl)piperidine-4-carbonyl]isoxazolidin-3-yl]benzonitrile,
3-fluoro-5-[(3S)-2-[1-(6-methoxypyrimidin-4-yl)piperidine-4-carbonyl]isoxazolidin-3-yl]-2-methyl-benzonitrile and
[(3S)-3-(3-chloro-5-fluoro-phenyl)isoxazolidin-2-yl]-[1-(6-methoxypyrimidin-4-yl)-4-piperidyl]methanone,
or a pharmaceutically acceptable salt, solvate, or stereoisomer thereof.

11. A pharmaceutical composition comprising a compound of any one of claims 1 to 10 and at least one pharmaceutically acceptable carrier.

12. A pharmaceutical composition comprising a compound of any one of claims 1 to 10, and at least one pharmaceutically acceptable carrier and/or one or more additional active pharmaceutical agent selected from a thrombolytic agent, a tissue plasminogen activator, an anticoagulant, a platelet aggregation inhibitor, an antimicrobial agent (an antibiotic, a broad-spectrum antibiotic, a lactam, an antimycobacterial agent, a bactericidal antibiotic, anti-MRSA therapy), a long acting beta agonist, a combination of an inhaled corticosteroid and a long acting beta agonist, a short acting beta agonist, a leukotriene modifier, an anti-IgE, a methylxanthine bronchodilator, a mast cell inhibitor, a protein tyrosine kinase inhibitor, a CRTH2/D prostanoid receptor antagonist, an epinephrine inhalation aerosol, a phosphodiesterase inhibitor, a combination of a phosphodiesterase-3 inhibitor and a phosphodiesterase-4 inhibitor, a long-acting inhaled anticholinergic, a muscarinic antagonist, a long-acting muscarinic antagonist, a low dose steroid, an inhaled corticosteroid, an oral corticosteroid, a topical corticosteroid, anti-thymocyte globulin, thalidomide, chlorambucil, a calcium channel blocker, a topical emollient, an ACE inhibitor, a serotonin reuptake inhibitor, an endothelin-I receptor inhibitor, an anti-fibrotic agent, a proton-pump inhibitor, a cystic fibrosis transmembrane conductance regulator potentiator, a mucolytic agent, pancreatic enzymes, a bronchodilator, an opthalmalic intravitreal injection, an anti-vascular endothelial growth factor inhibitor, a ciliary neurotrophic growth factor agent, a trivalent (IIV3) inactivated influenza vaccine, a quadrivalent (IIV4) inactivated influenza vaccine, a trivalent recombinant influenza vaccine, a quadrivalent live attenuated influenza vaccine, an antiviral agent, inactivated influenza vaccine, a ciliary neurotrophic growth factor, a gene transfer agent, a topical immunomodulator, calcineurin inhibitor, an interferon gamma, an antihistamine, a monoclonal antibody, a polyclonal anti-Tcell antibody, an anti-thymocyte gamma globulin-equine antibody, an antithymocyte globulin-rabbit antibody, an anti-CD40 antagonist, a JAK inhibitor, and an anti-TCR murine mAb.

13. The compound according to any one of claims 1 to 10, or a pharmaceutical composition according to any one of claims 11 to 12 for the use in therapy.

14. The compound according to any one of claims 1 to 10, or a pharmaceutical composition according to any one of claims 11 to 12, for use in the treatment of a RIP kinase 1 mediated disease or disorder.

15. The compound according to any one of claims 1 to 10, or a pharmaceutical composition according to any one of the claims 11 to 12, for use in the treatment of necrotizing enterocolitis, tuberous sclerosis, Tangier's Disease, Wohlman's Syndrome, inflammatory bowel disease, Crohn's disease, ulcerative colitis, psoriasis, retinal detachment, retinitis pigmentosa, macular degeneration, pancreatitis (e.g., acute pancreatitis), atopic dermatitis, rheumatoid arthritis, psoriatic arthritis, spondyloarthritis, gout, SoJIA, systemic lupus erythematosus, Sjogren's syndrome, systemic scleroderma, anti-phospholipid syndrome, vasculitis, osteoarthritis, non-alcohol steatohepatitis, alcohol steatohepatitis, autoimmune hepatitis autoimmune hepatobiliary diseases, primary sclerosing cholangitis, nephritis, Celiac disease, autoimmune ITP, transplant rejection, ischemia, ischemia reperfusion injury of solid organs, cerebral ischemia, sepsis, systemic inflammatory response syndrome, cerebrovascular accident, myocardial infarction, Huntington's disease, Alzheimer's disease, Parkinson's disease, allergic diseases, asthma, atopic dermatitis, multiple sclerosis, type I diabetes, Wegener's granulomatosis, pulmonary sarcoidosis, Behçet's disease, interleukin-1 converting enzyme associated fever syndrome, chronic obstructive pulmonary disease, tumor necrosis factor receptor-associated periodic syndrome, periodontitis, infection, bacterial infection, staphylococcus infection, mycobacterium infection, influenza, transplant rejection, burns, hypoxia, trauma, stroke, cardiac infarction, lysosomal storage disease, Niemann-Pick disease, Gaucher's disease, Krabbe disease, amyotrophic lateral sclerosis (ALS), HIV-associated dementia, encephalopathy, retinal degenerative disease, glaucoma, age-related macular degeneration, Friedreich's ataxia, Lewy body disease, diabetic neuropathy, polyglutamine (polyQ) diseases, Fahr disease, Menke's disease, Wilson's disease, a prion disorder, atherosclerosis, Guillain-Barre syndrome, chronic inflammatory demyelinating polyneuropathy, major depressive disorder, bipolar disorder, delirium, post-operative cognitive impairment, autism, schizophrenia, hidradenitis suppurativa or incontinentia pigmenti.

16. The compound according to any one of claims 1 to 10, or a pharmaceutical composition according to any one of the claims 11 to 12, for use in the treatment of Alzheimer disease, multiple sclerosis, amyotrophic lateral sclerosis (ALS) and incontinentia pigmenti.
